(19) United States
(12) Patent Application Publication
(10) Pub. No.: US 2019/0144481 A1
(43) Pub. Date: May 16, 2019

(54) PROCESSED STACHYOSE COMPOSITIONS

(71) Applicant: EPC Natural Products Co., Ltd., Beijing (CN)

(72) Inventors: Hansheng Wang, Beijing (CN); Jingang Shi, Beijing (CN)

(21) Appl. No.: 16/157,489

(22) Filed: Oct. 11, 2018

Related U.S. Application Data
(60) Provisional application No. 62/573,388, filed on Oct. 17, 2017.

(51) Int. Cl. C07H 3/06; A23L 33/105

(52) U.S. Cl. CPC C07H 3/06; A23L 33/105; A23V 2250/06; A23V 2200/3202; A23V 2250/284; A23V 2002/00

(57) ABSTRACT

The invention describes stachyose compositions and methods to prepare such compositions with reduced or eliminated odor causing impurities, such as aldehydic impurities, contained within stachyose compositions that occur in nature or are caused during commercial processing.

### CROSS REFERENCE TO RELATED APPLICATIONS

This application claims priority to U.S. Provisional Patent Application No. 62/573,388, filed Oct. 17, 2017, the disclosure of which is hereby expressly incorporated by reference in its entirety.

### FIELD OF THE INVENTION

The embodiments described herein relate generally to compositions of oligosaccharides including stachyose, melitriose, verbascose and mixtures thereof and methods to prepare the oligosaccharides. In one aspect, the stachyose, melitriose, verbascose or mixtures thereof do not include odor inducing components such as aldehydes, including but not limited to isobutyl aldehyde, isovaleraldehyde and/or 2-methyl-butyl aldehyde and mixtures thereof. Amino acids and/or salts, as well as prebiotics and/or probiotics, can be added to the oligosaccharide compositions to eliminate or reduce odiferous impurities to provide a pleasant tasting composition.

### BACKGROUND OF THE INVENTION

Stachyose is present in various plants including beans with about 2˜4% stachyose being present. Stachyose is also present in Chinese herbs, such as Rehmannia Glutinosa, Eupatorium Japonicum Thumb and common vegetables, such as green beans, soy beans, peas and Chinese artichoke, known as Stachys affinis or Stachys Sieboldii.

Stachyose is also present in Devil's Claw, which is Harpagophytum species, including Harpagophytum procumbens, and Harpagophytum zeyheri Decne. As for this plant, the current focus in food supplement industry is to enrich Harpagpside for joint-health. There is less attention to be paid for taking full use of nutritional value of Devil's Claw.

The major constituents of the Devil's Claw are iridoid glycosides, sugars, triterpenoids, phytosterols and aromatic acids. In addition to stachyose, the Devil's Claw further contains some carbohydrates such as fructose, glucose, raffinose, and sucrose, diterpenes such as 8, 11, 13-totaratriene-12, 13-diol, 8, 11, 13-abietatrien-12-ol, iridoids such as harpagide, harpagoside, and other constituents such as acetoside, isoacteoside, flavonoids, etc.

Stachyose, is considered a prebiotic and is an oligosaccharide which can help promote multiplication of probiotics, such as Bifidobacterium, in the intestine, thus helping with the digestion of food and nutrients.

Stachyose is a tetrasaccharide consisting of two α-D-galactose units, one α-D-glucose unit, and one β-D-fructose unit sequentially linked as gal(α1→6)gal(α1→6)glc(α1↔2β)fru.

Stachyose is less sweet than sucrose, at about 28% on a weight basis (e.g., the sweetness of 100 grams of stachyose is equal to that of 28 grams of sucrose). It is mainly used as a bulk sweetener or for its functional oligosaccharide properties. Stachyose is not completely digestible by humans and delivers 1.5 to 2.4 kcal/g (6 to 10 kJ/g).

Stachyose has a molecular formula of C₂₄H₄₂O₂₁, M.W. 666.59 and a melting point of 167-170° C. The structural formula of stachyose is

Stachyose is known by its IUIPAC name as (2R,3R,4S,5R,6R)-2-[(2S,3S,4S,5R)-3,4-Dihydroxy-2,5-bis(hydroxymethyl) oxolan-2-yl]oxy-6-[[(2S,3R,4S,5R,6R)-3,4,5-trihydroxy-6-[[(2S,3R,4S,5S,6R)-3,4,5-trihydroxy-6-(hydroxymethyl)oxan-2-yl]oxymethyl] oxan-2-yl]oxymethyl]oxane-3,4,5-triol and also by β-D-Fructofuranosyl-O-α-D-galactopyranosyl-(1→6)-O-α-D-galactopyranosyl-(1→6)-α-D-glucopyranoside.

Commercially available stachyose often has a pungent odor which can make it smell unpleasant, have a poor taste and can be unappealing to the consumer.

Therefore, a need exists to provide stachyose compositions that overcome one or more of the current disadvantages noted above.

### BRIEF SUMMARY OF THE INVENTION

In one aspect, the oligosaccharide compositions of either stachyose or stachyose, raffinose and verbascose further include one or more amino acid(s), metal salt(s), additional prebiotic(s), probiotics or mixtures thereof, still while retaining the pleasant smell and taste not typically associated with traditional and commercial stachyose products.

In still another aspect, the oligosaccharide compositions of either stachyose or stachyose, raffinose (melitriose) and verbascose are presented that do not contain odiferous aldehydes, such as 2-methyl butylaldehyde, isovaleraldehyde, isobutyl aldehyde or mixtures thereof. These oligosaccharide compositions are devoid of odor(s) and/or do not have a bad taste due to impurities in the components of the oligosaccharide composition.

In yet another aspect, it has surprisingly been found that stachyose compositions or stachyose, raffinose and verbascose compositions that contain odiferous impurities, such as aldehydes, can have the smell and/or poor taste masked by addition of a sufficient amount of one or more of amino acid(s) or metal salts. These compositions can further include one or more prebiotic(s) and/or probiotic(s).

In still yet another aspect, methods to provide oligosaccharide containing stachyose compositions or stachyose, raffinose and verbascose oligosaccharide compositions devoid of odiferous material(s) is detailed such that the oligosaccharide compositions do no possess a foul smell, have a pleasant taste and/or are devoid of aldehydic impurities.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

In still another aspect, the inventors developed a purified Stachyose from Devil's Claw which could be used in the food, beverage, feed, cosmetic and pharmaceutical industry. The content of Stachyose in the Devil's Claw extract could be above 40%, preferably 50%, 60%, 70%, 80%, 90%, 99%. Dosage values of using such purified extract in food, beverage, feed, cosmetic and pharmaceutical industry could be in range of 0.1˜50 gram/day, preferably 1˜10 gram/day.

### BRIEF DESCRIPTION OF THE DRAWINGS

FIG. 1 depicts the standard curve of the analysis on total sugar content in example 1.

FIG. 2 depicts the standard curve of the analysis on stachyose content in example 2.

FIG. 3 depicts the standard curve of the analysis on reducing sugar content in example 3.

FIG. 4 depicts the ions content of sample 1 in example 4.

FIG. 5A depicts the amino acid content analysis of sample 2 in example 5.

FIG. 5B depicts the amino acid content analysis of sample 3 in example 5.

FIG. 5C depicts the amino acid content analysis of sample 1 in example 5.

FIG. 6 depicts the standard curve of the analysis on 2-methyl butylaldehyde content in example 6.

FIG. 7 depicts the standard curve of the analysis on isovaleraldehyde content in example 6.

FIG. 8 depicts the standard curve of the analysis on isobutyl aldehyde content in example 6.

FIG. 9 depicts the GCMS-carbon powder overlay and comparison graph of the reference substance prepared in example 6 and samples of present embodiments.

FIG. 10 depicts the GCMS-carbon powder graph of the reference substance prepared in example 6.

FIG. 11 depicts the MS graph of the reference substance prepared in example 6 at 5.12 8 min.

FIG. 12 depicts the MS graph of the reference substance prepared in example 6 at 5.317 min.

FIG. 13 depicts the MS graph of the reference substance prepared in example 6 at 6.716 min.

FIG. 14 depicts the MS graph of the reference substance prepared in example 6 at 7.099 min.

FIG. 15 depicts the MS graph of the reference substance prepared in example 6 at 7.434 min.

FIG. 16 depicts the MS graph of the reference substance prepared in example 6 at 9.244 min.

FIG. 17 depicts the MS graph of the reference substance prepared in example 6 at 9.568 min.

FIG. 18 depicts the MS graph of the reference substance prepared in example 6 at 10.616 min.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Commercially available stachyose has an unpleasant smell and/or taste due to impurities from the plant source. Isolation of the stachyose without an odiferous smell has not been achieved until the time of the present embodiments.

The present embodiments, in one aspect, utilize unique isolation and purification processes from plant material to provide stachyose or compositions of stachyose with raffinose and/or verbascose.

The present embodiments surprisingly provide oligosaccharide compositions comprising stachyose that is devoid of odor(s) and/or does not have a bad taste due to impurities in the stachyose.

In another embodiment, an oligosaccharide composition of stachyose with raffinose (melitriose) and verbascose is provide that is also devoid of odor(s) and/or does not have a bad taste due to impurities in the components of the oligosaccharide composition.

Stachyose, raffinose and verbascose are oligosaccharides that can serve as prebiotics and are non-digestible short-chain carbohydrates. Humans do not have enzymes to digest them, so they pass unchanged to the colon where the normal intestinal bacteria ferment them.

In the large intestine, raffinose, stachyose and verbascose act as a soluble dietary fiber.

Raffinose, also called melitose or melitriose, is composed of 3 sugars: galactose, glucose and fructose. Examples of edible plants naturally high in raffinose are beans, asparagus, cotton seeds, sugar beet molasses, cabbage, broccoli, Brussel's sprouts, sweet potatoes and whole grains.

Stachyose is composed of 4 sugar molecules: 2 galactoses, glucose and fructose. It is found mainly in legumes, such as beans and peas.

Verbascose is composed of five sugar molecules: 3 galactoses, glucose and fructose and is found in legumes.

It has been found that stachyose or a combination of stachyose, melitriose and verbascose can be isolated from Chinese artichoke, Rehmannia Glutinosa, Eupatorium Japonicum Thumb and common vegetables, such as green beans, soy beans, and peas.

Probiotics are types of ‘living’ friendly bacteria similar to those that inhabit our digestive tract. They are naturally found in cultured or fermented foods such as yogurt, buttermilk, aged cheese, sauerkraut, sourdough bread, miso, tempeh and kombucha a type of fermented tea and can also be taken in supplement form. Probiotics help to maintain healthy levels of good bacteria in the intestines, they support our immune defenses, are useful for anyone suffering from the uncomfortable symptoms of bloating, gas or flatulence and may assist in decreasing the duration of diarrhea in children. They may also help to restore good bacteria after a course of antibiotics. There are many different types of probiotics and each behaves a little differently. Lactobacillus acidophilus and bifidibacterium lactis, for example, are probiotics which are often found in yogurt.

Prebiotics are ‘non-living’ food ingredients that reach the large intestine unaffected by digestion, and ‘feed’ the good bacteria in the gut by helping the bacteria, such as lactobacillus and/or bifidibacterium, to grow and flourish. Prebiotics such as fructooligosaccharides (FOS) and galacto-oligosaccharides (GOS) are naturally found in many foods including: legumes, whole wheat products, rye based foods, artichokes, onions, cabbage, garlic and chicory root which contains inulin.

Other prebiotics include, for example, arabinose, galactose, inulin, raffinose, mannose, lactulose, mannanoligosaccharides, xylooligosaccharides, palatinose, lactosucrose, glycooligosaccharides, isomaltooligosaccharides, polydextrose, or soybean oligosaccharides.

Having a combination of prebiotic and probiotic rich foods and ingesting a supplement if needed can help an individual maintain a healthy balance of good bacteria and support health and wellbeing. They may be especially beneficial for active growing bodies to support their developing digestive and immune systems and to help relieve intestinal issues such as diarrhea.

Commercial probiotic preparations are usually mixtures of lactobacilli and Bifidobacterium, although yeasts such as saccharomyces have also been used. Bifidobacteria are of particular interest. These are anaerobic pleomorphic rods or club shaped organisms which normally have an important role in breaking down dietary carbohydrate and interact directly with the host metabolism. Bifidobacteria also synthesize and excrete water soluble vitamins, but there are considerable differences in species and strains. These organisms predominate in the colons of breastfed babies; they account for up to 95% of all culturable bacteria and protect against infection. Bifidobacteria do not occur in such high numbers in adults.

As noted above, one issue with available compositions containing stachyose is the odor associated with the material. Therefore, embodiments disclosed herein provide methods to remove or reduce the offending smell and associated poor taste from stachyose compositions.

Stachyose compositions, including those that contain raffinose and/or verbascose are used as additives in food products. Thus, the removal of offending impurities from the oligosaccharides is advantageous to the supplier and the consumer. Thus, the stachyose compositions described herein can be incorporated into consumables, including but not limited to food products, flavorings, pharmaceutical compositions, dietary supplements, nutraceuticals, beverages, etc.

For example, food products include, but are not limited to, cereal products, rice products, tapioca products, sago products, baker's products, biscuit products, pastry products, bread products, confectionery products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yogurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof.

The stachyose compositions disclosed herein are effective to help promote digestion and maintenance of gut flora once ingested, even in relatively small amounts. In one aspect, the average daily intake (ADI) is below 15 g/day, more particularly from about 0.1 g and 12 g/day, and more particularly the ADI is between Ig and 12 g/day, or 3 g and 10 g/day, or 0.5 g and 10 g/day.

It should be understood that throughout this specification that “stachyose” composition can include only stachyose (consisting of) or stachyose and other components, such as melitriose, verbascose or mixtures thereof. Therefore, when the term “stachyose composition” is noted, it should not be considered limited to only stachyose unless specifically noted as such.

There are two important aspects related to the present embodiments. Both provide advantages over current available technology.

In one aspect, the stachyose composition is not odiferous or has the offending material reduced significantly or eliminated so that offending smells are not noticeable by an individual and the smell and taste profile of the present composition is an improvement those stachyose compositions currently available.

In another aspect, the stachyose composition is not odiferous, as noted above, and further comprises one or more of an amino acid(s), a metal salt(s), a prebiotic (other than what is present in the stachyose composition), a probiotic or mixtures thereof.

In one aspect, the non-odiferous stachyose composition is devoid of aldehydic impurities. Examples of aldehydic impurities include one or more of isobutyl aldehyde, isovaleraldehyde, 2-methyl butyl aldehyde or mixtures thereof.

In an aspect, the amount of aldehydic impurity left in the stachyose composition is not detectable by gas chromatography (GC) or high performance liquid chromatography (HPLC). In another aspect, the total amount of aldehydic impurity(ies) is below about 3 ppm, below about 2.5 ppm, below about 2.2 ppm, below about 2 ppm, below about 1.5 ppm, below about 1.3 ppm, more particularly below about 1.2 ppm, even more particularly below about 1 ppm and most particularly below about 0.5 ppm, below 0.3 ppm, below 0.2 ppm, below 0.1 ppm and below 0.05 ppm, below a trace amount and most particularly, about 0 ppm.

In another aspect, the stachyose composition may have offensive smell(s) or tastes, but these can be overcome by the addition of one or more additives, such as amino acid(s) and/or metal salt(s). The stachyose (with the offending smell and/or taste) is treated with a sufficient amount of either or both amino acid(s) and/or metal salt(s) to reduce or eliminate the offending material(s).

In still another embodiment, the stachyose composition that may have offensive smell(s) or taste, can further comprise one or more of a prebiotic(s) and/or probiotic(s), more particularly in combination with a smell reducing/taste improving quantify of amino acid(s) and/or metal salt(s).

Suitable amino acids include, for example, arginine, glutamic acid, threonine, aspartic acid, lysine, valine, serine, alanine, glycine, proline, leucine, isoleucine, histidine or mixtures thereof.

Stachyose compositions can include from about 92% to about 99.5% by weight total oligosaccharide(s) with an amino acid content of from about 0.5% to about 8% by weight.

In another aspect, the stachyose compositions include about 95% by weight total oligosaccharide(s) with an amino acid content of about 5% by weight.

In still another aspect, the content of the stachyose in the oligosaccharide composition with amino acid(s) is from about 55% to about 94% by weight or from about 70% to about 90% by weight.

Suitable metal salts include alkali and alkaline-earth metal salts. The metals and anions include, for example K⁺, Ca²⁺, Cu²⁺, Cu⁺, Na⁺, Mg²⁺, Mn²⁺, Mo³, Fe²⁺, Fe³⁺, or Al³⁺ and mixtures thereof, and PO₄³⁻, S₄²⁻, NO₃⁻, Cl⁻, Br⁻, I⁻, CO₃⁻², or HCO₃⁻ and mixtures thereof. For example, KCl, NaCl, KOH, NaOH, NaHCO₃, KHCO₃, etc.

Stachyose compositions can include from greater than about 85% by weight total oligosaccharide(s) with metal salt content of from less than about 15% by weight.

Stachyose compositions can include from about 90% to about 99.5% by weight total oligosaccharide(s) with metal salt content of from about 0.5% to about 10% by weight.

In another aspect, the stachyose compositions include about 95% by weight total oligosaccharide(s) with a metal salt content of about 5% by weight.

In still another aspect, the content of the stachyose in the oligosaccharide composition with metal salt(s) is from about 55% to about 94% by weight or from about 70% to about 90% by weight.

In still another aspect, the total oligosaccharide content of the stachyose composition with amino acid(s) and metal salt(s) is from greater than about 75% by weight, the amino acid(s) content is from less than about 10% by weight and the metal salt(s) content is from less than about 15% by weight.

In yet another aspect, the total oligosaccharide content of the stachyose composition with amino acid(s) and metal salt(s) is from about 82% to about 89% by weight, the amino acid(s) content is from about 0.5% to about 8% by weight and the metal salt(s) content is from about 0.5% to about 10% by weight.

In another aspect, the stachyose compositions include about 90% by weight total oligosaccharide(s) with an amino acid(s) content of about 5% by weight and a metal salt content of about 5% by weight.

In still another aspect, the content of the stachyose in the oligosaccharide composition with amino acid(s) and metal salt(s) is from about 55% to about 94% by weight or from about 70% to about 90% by weight.

In one aspect, a stachyose containing plant material is subjected to a process to provide an isolated stachyose composition. Suitable sources for the stachyose include those previously noted as well as Chinese articoke (Stachys affinis), also known as crosne, Japanese artichoke, knotroot and artichoke betony or Stachy sieboldii Miq, or Stachys flordana. Generally the tuber (tuberous portion) of the plant is utilized for the processing of the stachyose containing material.

The plant material is mixed with water, ground up/smashed, and a first thick liquid (a juice) is obtained. Generally, the ratio of plant material to water is approximately 4:1 to about 0.5:1 by weight. This step is generally carried out at ambient temperature and the time of mixing/grinding/smashing is from about 10 minutes to about 2 hours, for example about 30 minutes to about 90 minutes, from about 15 minutes to about 100 minutes, from about 45 minutes to about 60 minutes and all values there between.

The first thick liquid is filtered. The remaining solid residue is treated again with the same amount of water as above and a second thick liquid is obtained. This second thick liquid is filtered and the filtrates of the first and second thick liquids are combined.

The filtrates are filtered through an ultrafiltration membrane having a MW cut off of 5000-10,000 Daltons to provide a filtered solution.

The filtered solution is treated with a base until the pH of the solution is between a pH of 9 to about 12 to afford a basified solution. Typical bases utilized for this step include, for example, calcium oxide, calcium hydroxide or sodium hydroxide. Generally the basified solution is maintained at a temperature of from about 20° C. to about 50° C. for between about 2 to about 3 hours.

The basified solution is filtered through a paper filter, a cloth or separated by centrifugation and then the filtered basified solution is heated without stirring from about 20° C. to about 50° C. for between about 2 to about 4 hours.

The basified filtrate is filtered through a paper filter, a cloth filter or centrifuged and the pH of the resulting filtrate is adjusted with an acid, such as for example sulfuric acid or phosphoric acid, until the pH of the filtrate is between about 4 and about 7. The acidified filtrate is allowed to remain at room temperature for between about 1 to about 2 hours.

If any insoluble substance is noted in the acidified filtrate, it is removed by filtration such as through a paper filter, cloth or centrifugation. The acidified filtrate is treated with activated carbon for about 30 minutes to about 3 hours at a temperature range of from about 20° C. to about 50° C. prior to filtration at a temperature range of from about 20° C. to about 50° C. The amount of activated carbon to the total filtrate is from about 0.5% to about 2% w/v (activated carbon to filtrate).

Generally, when the filtrates note above are treated with base, the pH of the filtrate is from about a pH of 7.5 to about 14, from about 8 to about 13, from about 9 to about 12, from about 10 to about 11 and all ranges noted there between.

Generally, when the filtrates note above are treated with acid, the pH of the filtrate is from about a pH of 1 to about 7, from about 4 to about 7, from about 5 to about 7, from about 5 to about 6 and all ranges noted there between.

Generally, when the filtrates or solutions are heated in the processes described herein, the temperature range is from about 30° C. to about 100° C., from about 40° C. to about 90° C., from about 50° C. to about 80° C., from about 60° C. to about 70° C., more particularly from about 40° C. to about 50° C. or from about 70° C. to about 90° C. and all ranges noted there between.

When mixtures, solutions or filtrates are stirred, such times are from about 5 minutes to about 3 days, from about 15 minutes to about 2 days, from about 30 minutes to about 24 hours, from about 1 hour to about 12 hours, from about 16 hours to about 18 hours, more particularly about 30 minutes to about 3 hours and all ranges noted there between.

The acidified and carbon treated filtrate is then spray dried to provide a powder. Although spray drying is known in the art, an L-117 lab spray dryer from Laiheng Lab-Equipment (China) was utilized with a temperature inlet of about 110° C. to about 220° C. with a pressure of about 5 to about 10 bar.

The powder obtained from spray drying is solubilized with a solvent such as water or ethanol, heated and then cooled to provide a precipitate. When ethanol is used, the ethanol content is from about 60 to about 90% ethanol with the remainder water. Generally, the volume of ethanol used is approximately 3 to about 5 times the weight of the powder, e.g., 3 ml ethanol to 1 gram of powder. Generally when water is used, the volume of water used is approximately from about to about 5 times the weight of the powder. The solution is heated from about 70° C. to about 80° C. until the powder is dissolved. The heated solution is then cooled to between about 0° C. to about 25° C. to provide the precipitate.

The precipitate is collected by filtration, as described above, washed with additional solvent, such as 95% ethanol or water, air dried or dried in a vacuum oven to provide a high purity stachyose composition that is free from odiferous materials, such as the aldehydes noted herein. Alternatively, the spray dried powder is dissolved in a solvent, such as water, and subjected to steam distillation, and the residue solution is then passed through a membrane and is spray dried to obtain a stachyose composition as a powder that is free from odiferous materials, such as the aldehydes noted herein.

In another embodiment, an amino acid can be added to the isolated stachyose composition in water and subjected to steam distillation. The residue is passed through a membrane and concentrated with spray drying to produce a stachyose composition that contains one or more amino acids.

The processes described herein provide a stachyose composition that includes from about 92 to about 99% by weight of stachyose, from about 0.5 to about 5% by weight melitriose and from about 0.5 to about 3% by weight of verbascose, and in particular, devoid of foul smelling impurities, such as aldehydes.

In other aspects of the invention, the stachyose composition can be further refined so that the percentage of stachyose is from about 99% by weight to 100% by weight, e.g., 99.5%, 99.6%, 99.7%, 99.8%, 99.9%, 99.99% by weight, and in particular, devoid of foul smelling impurities, such as aldehydes disclosed herein.

It should be understood that the stachyose oligosaccharide compositions described herein can be obtained by the methods of preparation detailed herein.

The following paragraphs enumerated consecutively from 1 through 64 provide for various aspects of the present invention. In one embodiment, in a first paragraph (1), the present invention provides an oligosaccharide composition comprising stachyose devoid of odor.

2. An oligosaccharide composition comprising stachyose, wherein one or more odiferous aldehydes are not present.

3. The oligosaccharide composition of paragraph 2, wherein the one or more odiferous aldehydes that are not present comprise isobutyl aldehyde, isovaleraldehyde, 2-methyl butyl aldehyde or mixtures thereof.

4. The oligosaccharide composition of any of paragraphs 1 through 3, further comprising oligosaccharide melitriose (raffinose) or verbascose or mixtures thereof.

5. The oligosaccharide composition of paragraph 4, further comprising an amino acid.

6. The oligosaccharide composition of paragraph 5, wherein the amino acid is one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or mixtures thereof.

7. The oligosaccharide composition of paragraph 6, wherein the oligosaccharide(s) are present from about 92% to about 99.5% by weight total oligosaccharide(s) with an amino acid content of from about 0.5% to about 8% by weight.

8. The oligosaccharide composition of paragraph 6, wherein the oligosaccharide(s) are present in about 95% by weight total oligosaccharide(s) with an amino acid content of about 5% by weight.

9. The oligosaccharide composition of paragraph 6, wherein the stachyose content in the oligosaccharide composition with amino acid(s) is from about 55% to about 94% by weight or from about 70% to about 90% by weight.

10. The oligosaccharide composition of paragraph 4, further comprising a metal salt.

11. The oligosaccharide composition of paragraph 10, wherein the metal salt comprises one or more anions comprising PO₄³⁻, SO₄²⁻, NO₃⁻, Cl⁻, Br⁻, I⁻, CO₃⁻², OH, or HCO₃⁻ and one or more cations comprising K⁺, Ca²⁺, Cu²⁺, Cu⁺, Na⁺, Mg²⁺, Mn²⁺, Mo³⁺, Fe²⁺, Fe³⁺, or Al³⁺.

12. The oligosaccharide composition of paragraph 11, wherein the oligosaccharide(s) are present in greater than about 85% by weight total oligosaccharide(s) with a metal salt content of from less than about 15% by weight.

13. The oligosaccharide composition of paragraph 11, wherein the oligosaccharide(s) are present from about 90% to about 99.5% by weight total oligosaccharide(s) with metal salt content of from about 0.5% to about 10% by weight.

14. The oligosaccharide composition of paragraph 11, wherein the oligosaccharide(s) are present in about 95% by weight total oligosaccharide(s) with an metal salt content of about 5% by weight.

15. The oligosaccharide composition of paragraph 11, wherein the stachyose content in the oligosaccharide composition with metal salt(s) is from about 55% to about 94% by weight or from about 70% to about 90% by weight.

16. The oligosaccharide composition of paragraph 4, further comprising one or more amino acid(s) and one or more metal salt(s).

17. The oligosaccharide composition of paragraph 16, wherein the total oligosaccharide content of the stachyose composition with amino acid(s) and metal salt(s) is from greater than about 75% by weight, the amino acid(s) content is from less than about 10% by weight and the metal salt(s) content is from less than about 15% by weight.

18. The oligosaccharide composition of paragraph 16, wherein the total oligosaccharide content of the stachyose composition with amino acid(s) and metal salt(s) is from about 82% to about 89% by weight, the amino acid(s) content is from about 0.5% to about 8% by weight and the metal salt(s) content is from about 0.5% to about 10% by weight.

19. The oligosaccharide composition of paragraph 16, wherein the stachyose compositions include about 90% by weight total oligosaccharide(s) with an amino acid(s) content of about 5% by weight and a metal salt content of about 5% by weight.

20. The oligosaccharide composition of paragraph 16, wherein the stachyose content in the oligosaccharide composition with metal salt(s) is from about 55% to about 94% by weight or from about 70% to about 90% by weight.

21. The oligosaccharide composition of paragraph 4, further comprising one or more prebiotics.

22. The oligosaccharide composition of paragraph 21, wherein the one or more prebiotic comprises fructooligosaccharides, galactooligosaccharides, arabinose, galactose, inulin, raffinose, mannose, lactulose, mannanoligosaccharides, xylooligosaccharides, palatinose, lactosucrose, glycooligosaccharides, isomaltooligosaccharides, polydextrose, or soybean oligosaccharides.

23. The oligosaccharide composition of paragraph 22, wherein the ratio of oligosaccharide to prebiotic is about 1:10, more particularly about 1:2, about 1:3, about 1:4, about 1:5 or about 1:6.

24. The oligosaccharide composition of paragraph 4, further comprising one or more probiotics.

25. The oligosaccharide composition of paragraph 24, wherein the one or more probiotics comprise Lactobacillus or Bifidobacteria.

26. The oligosaccharide composition of paragraph 25, wherein the ratio of oligosaccharide to probiotic is about 1:10, more particularly about 1:2, about 1:3, about 1:4, about 1:5 or about 1:6.

27. An oligosaccharide composition comprising stachyose, melitriose (raffinose) and verbascose.

28. The oligosaccharide composition of paragraph 27, further comprising a sufficient amount of an amino acid to reduce or eliminate aldehydic odor of the oligosaccharide composition.

29. The oligosaccharide composition of paragraph 28, wherein the amino acid is one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or mixtures thereof.

30. The oligosaccharide composition of paragraph 29, wherein the oligosaccharide(s) are present from about 92% to about 99.5% by weight total oligosaccharide(s) with an amino acid content of from about 0.5% to about 8% by weight.

31. The oligosaccharide composition of paragraph 29, wherein the oligosaccharide(s) are present in about 95% by weight total oligosaccharide(s) with an amino acid content of about 5% by weight.

32. The oligosaccharide composition of paragraph 6, wherein the stachyose content in the oligosaccharide composition with amino acid(s) is from about 55% to about 94% by weight or from about 70% to about 90% by weight.

33. The oligosaccharide composition of paragraph 27, further comprising a sufficient amount of a metal salt to reduce or eliminate aldehydic odor of the oligosaccharide composition.

34. The oligosaccharide composition of paragraph 33, wherein the metal salt comprises one or more anions comprising PO₄³⁻, SO₄²⁻, NO₃⁻, Cl⁻, Br⁻, I⁻, CO₃⁻², OH, or HCO₃⁻ and one or more cations comprising K⁺, Ca²⁺, Cu²⁺, Cu⁺, Na⁺, Mg²⁺, Mn²⁺, Mo³⁺, Fe²⁺, Fe³, or Al³⁺.

35. The oligosaccharide composition of paragraph 34, wherein the oligosaccharide(s) are present from about 90% to about 99.5% by weight total oligosaccharide(s) with metal salt content of from about 0.5% to about 10% by weight.

36. The oligosaccharide composition of paragraph 34, wherein the oligosaccharide(s) are present in about 95% by weight total oligosaccharide(s) with an metal salt content of about 5% by weight.

37. The oligosaccharide composition of paragraph 33, wherein the stachyose content in the oligosaccharide composition with metal salt(s) is from about 55% to about 94% by weight or from about 70% to about 90% by weight.

38. The oligosaccharide composition of paragraph 27, further comprising one or more amino acid(s) and one or more metal salt(s).

39. The oligosaccharide composition of paragraph 27, wherein the total oligosaccharide content of the stachyose composition with amino acid(s) and metal salt(s) is from greater than about 75% by weight, the amino acid(s) content is from less than about 10% by weight and the metal salt(s) content is from less than about 15% by weight.

40. The oligosaccharide composition of paragraph 27, wherein the total oligosaccharide content of the stachyose composition with amino acid(s) and metal salt(s) is from about 82% to about 89% by weight, the amino acid(s) content is from about 0.5% to about 8% by weight and the metal salt(s) content is from about 0.5% to about 10% by weight.

41. The oligosaccharide composition of paragraph 27, wherein the stachyose compositions include about 90% by weight total oligosaccharide(s) with an amino acid(s) content of about 5% by weight and a metal salt content of about 5% by weight.

42. The oligosaccharide composition of paragraph 27, wherein the stachyose content in the oligosaccharide composition with metal salt(s) is from about 55% to about 94% by weight or from about 70% to about 90% by weight.

43. The oligosaccharide composition of any of paragraphs 27 through 42, further comprising one or more prebiotics.

44. The oligosaccharide composition of paragraph 43, wherein the one or more prebiotic comprises fructooligosaccharides, galactooligosaccharides, arabinose, galactose, inulin, raffinose, mannose, lactulose, mannanoligosaccharides, xylooligosaccharides, palatinose, lactosucrose, glycooligosaccharides, isomaltooligosaccharides, polydextrose, or soybean oligosaccharides.

45. The oligosaccharide composition of paragraph 44, wherein the ratio of oligosaccharide to prebiotic is about 1:10, more particularly about 1:2, about 1:3, about 1:4, about 1:5 or about 1:6

46. The oligosaccharide composition of any of paragraphs 27 through 45, further comprising one or more probiotics.

47. The oligosaccharide composition of paragraph 46, wherein the one or more probiotics comprise Lactobacillus or Bifidobacteria.

48. The oligosaccharide composition of paragraph 46, wherein the ratio of oligosaccharide to probiotic is about 1:10, more particularly about 1:2, about 1:3, about 1:4, about 1:5 or about 1:6

49. The oligosaccharide compositions of any of paragraphs 1 through 48, wherein the composition further includes a food product, a flavoring, a pharmaceutical composition, a dietary supplement, or a nutraceutical.

50. The oligosaccharide compositions of paragraph 49, wherein the amount of stachyose provided on a daily basis is less than 15 g/day.

51. The oligosaccharide compositions of paragraph 49, wherein the amount of stachyose provided on a daily basis is between about 1 to about 12 g/day.

52. The oligosaccharide compositions of paragraph 49, wherein the amount of stachyose provided on a daily basis is between about 3 to about 10 g/day.

53. The oligosaccharide compositions of any of paragraphs 1 through 52, wherein the stachyose is derived from Chinese herb and/or common vegetable; preferably, the Chinese herb is selected from Rehmannia Glutinosa and/or Eupatorium Japonicum Thumb; and the common vegetable is selected from Chinese artichoke and/or legume; more preferably, the legume is selected from green beans, soy beans and/or peas.

54. The oligosaccharide compositions of any of paragraphs 4 through 53, wherein the melitriose is derived from Chinese herb and/or edible plant, preferably, the Chinese herb is selected from Rehmannia Glutinosa and/or Eupatorium Japonicum Thumb; and the edible plant is selected from the group consisting of Chinese artichoke, beans, asparagus, cotton seeds, sugar beet molasses, cabbage, broccoli, Brussel's sprouts, sweet potatoes and/or whole grains.

55. The oligosaccharide compositions of any of paragraphs 4 through 54, wherein the verbascose is derived from Chinese herb and/or edible plant; preferably, the Chinese herb is selected from Rehmannia Glutinosa and/or Eupatorium Japonicum Thumb; and the edible plant is selected from Chinese artichoke, and/or legume; more preferably, the legume is selected from green beans, soy beans and/or peas.

56. A method to prepare a stachyose composition devoid of odor, comprising the steps:

a) mixing a stachyose containing plant material with water to provide a mixture;

b) isolating a filtrate from the mixture;

c) treating the filtrate with a base until the pH of the filtrate is about 9 to about 10 to provide a basified solution which is heated to about 45° C.;

d) filtering the basified solution to remove any solids to provide a filtered basified filtrate;

e) treating the filtered basified filtrate with an acid until the pH of the filtered basified filtrate is about 6 to about 7 to afford an acidified filtrate;

f) filtering the acidified filtrate to provide a filtered acidified filtrate;

g) treating the filtered acidified filtrate with activated carbon, steam distillation or vacuum distillation to provide a final filtrate or residual solution in the distillation apparatus; and

h) removing liquid from the final filtrate or residual solution to provide a stachyose composition devoid of odor causing impurities.

57. A devil's claw extract comprising stachyose and harpagoside.

58. The devil's claw extract of paragraph 57, wherein the stachyose is present at an amount of greater than 50 wt %, preferably greater than 60 wt %, more preferably greater than 70 wt % in the extract, most preferably greater than 95 wt % in the extract.

59. The devil's claw extract of paragraph 57 or paragraph 58, wherein the harpagoside is present at an amount of less than 2.0 wt %, preferably less than 1.0 wt % in the extract.

60. The devil's claw extract of paragraph 57 or paragraph 58, wherein the devil's extract is absence of harpagoside.

61. A composition comprising the devil's claw extract of any of paragraphs 57-60 and the materials selected from one of a food product, a flavoring, a pharmaceutical composition, a dietary supplement, or a nutraceutical.

62. The composition of paragraph 61, wherein the amount of stachyose provided on a daily basis is from 0.1-50 g/day, preferably 1.0-30.0 g/day, more preferably 1.0-10.0 g/day.

63. A method for preparing the devil's claw extract of any of paragraphs 57-60, wherein the method comprises the steps of:

(a) Adding the devil's claw powder to extract solvent;

(b) Performing extraction under condition of reflux to provide the first extraction solution;

(c) Filtering the first extraction solution;

(d) The filtering cake was subjected to the process in step (b), obtaining the second extraction solution;

(e) Combining the first and the second extract solutions to provide a mixed extraction solution;

(f) Purifying the mixed extraction solution by macroporous adsorption resin;

(g) Concentrating and drying the purified solution to obtain the devil's claw extract.

64. The method of paragraph 63, wherein the extract solvent comprises water, ethanol or the combination thereof.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Example 1 Analysis on Total Sugar Content

Anthrone colorimetry method was adopted to determine the content of total sugar, the result is calculated on the content of glucose.

1.1 Solution Preparation

75% sulfuric acid: 225 ml concentrated sulfuric acid was added to 75 ml water and cooled.

Anthrone solution: 0.2 g Anthrone was added to 100 ml 75% sulfuric acid.

0.6 mol/L HCl solution: 5 ml concentrated hydrochloric acid was added to 95 ml H2O.

2.4 mol/L HCl solution: 20 ml concentrated hydrochloric acid was added to 80 ml H2O.

1.2 mol/L H2SO4 solution: 6.5 ml concentrated sulfuric acid was added to 95 ml water.

1.2 Experiment

1.2.1 Testing of Pretreatment Conditions

1.2.1.1 Whether Sample could be Hydrolyzed or not

55 mg sample stachyose (Xi'An App-Chem Bio(Tech) Co., Ltd. China) was added to a 100 ml Florence flask with 25 ml 0.6 mol/L HCl. The solution was heated to reflux for 15 min at 90° C. in a water bath. The mixture was cooled to room temperature and water was added to the 100 ml volumetric flask to equal 100 ml. 3 ml of the solution was measured and added into a 25 ml volumetric flask and brought to volume by addition of water. 1 ml solution to be measured was added to a 10 ml color-comparison tube with a plug (inserting into ice bath). 6 ml Anthrone solution was added to the tube, mixed and reacted for 16 min in a boiled-water bath, after which the tube was placed immediately into an ice bath, and the absorbance of the solution was measured at 620 nm, with water as the blank.

Results:

| Sample No. | Way of processing | Abs/mg |
|---|---|---|
| 1 | 0.6MHCl | 5.890 |
| | water (un-reflux) | 5.351 |
| | water (reflux under | 5.411 |
| | 90° C.) | |

The data above showed that the stachyose required processing with the addition of acid and reflux prior to undertaking measurement(s).

1.2.1.2 Different Acids

55 mg sample product stachyose (Xi'An App-Chem Bio(Tech) Co., Ltd. China) was added to 100 ml Florence flask in duplicate. 25 ml 2.4 mol/L HCl and 1.2 mol/L H₂SO₄ were added respectively to the flasks and filled to 100 ml with water. The solutions were heated to reflux for 30 min at 90° C. in a water bath. The samples were cooled to room temperature, and Absorbance (Abs) was measured as described in 1.2.1.1.

Results:

| Sample No. | acid (C(H)⁺ = 2.4 mol/L) | Abs/mg |
|---|---|---|
| 1 | HCl | 5.81 |
| | H₂SO₄ | 5.00 |

According to the result above, absorbance was higher after preprocessing with HCl. This showed that preprocessing with HCL would have a better hydrolyzation than preprocessing with H₂SO₄. This showed it was acceptable to utilize HCl in the pretreatment process and the data were measured by adding HCl and refluxing.

1.2.1.3 Reference Substance Recycling Experiments

A given amount of glucose was added to a 100 ml Florence flask with 25 ml 0.6 mol/L HCl, refluxed for 15 min at 90° C. in a water bath, and the mixture was cooled to room temperature. Water was added to the 100 ml volumetric flask and brought to volume. 5 ml of the solution was added into a 50 ml volumetric flask and water was added to volume.

A blank (just 25 ml 0.6 mol/L HCl) was prepared with the same method as above. Absorbance as detailed in 1.2.1.1 was determined and the recycling rate of the reference substance was calculated.

Results:

| | | Recycling |
|---|---|---|
| Sample product | Quality/mg | rate (%) |
| glucose | 55.01 | 101.8 |
| | 34.82 | 100.7 |

The data above show that it was feasible to measure total sugar content (calculated on the basis of glucose) after hydrolysis of a product.

1.2.2 Measurements on the Sample Products (According to Examples 8 through 12)

1.2.2.1 Standard Curve

50 mg glucose was added into a 50 ml volumetric flask and brought to volume by addition of water with ultra-sonication. 0.2 ml, 0.4 ml, 0.6 ml, 0.8 ml of the solution was added respectively to 10 ml volumetric flasks and brought to volume by addition of water. The absorbance (ABS) was measured for each of them.

1.2.2.2 Sample Product Solution

60 mg of a sample, as noted below, was added to a 100 ml Florence flask with 25 ml 0.6 mol/L HCl, and refluxed for 15 min at 90° C. in a water bath. The mixture was cooled to room temperature and brought to volume with the addition of water. 5 ml of the solution was added to a 50 ml volumetric flask and brought to volume by addition of water. 1 ml of the solution to be measured was added to 10 ml color-comparison tube with a plug (inserting into ice bath). 6 ml Anthrone solution was added to the tube, mixed and reacted for 16 min in a boiling-water bath followed by placing the tube and its contents immediately into an ice bath. A blank (just 25 ml 0.6 mol/L HCl) was prepared with the same method as above and the absorbance of the solution was measured at 620 nm.

1.2.2.3 Calculation

$$total sugar \% = \frac{( A - b ) * f * 0.9 * 100}{k * m}$$

$$A \text{:} Abs$$

$$b \text{:} intercept of standard curve$$

$$f \text{:} dilution multiple of sample product$$

$$k \text{:} slope of standard curve$$

$$m \text{:} quality of sample product , mg$$

1.2.2.4 Results

| Concentration of | | | |
|---|---|---|---|
| mother liquor of | Concentration of | | |
| reference | diluent of reference | | Formula of |
| substance(mg/ml) | substance(mg/ml) | Abs | Standard curve |
| 1.0518 | 0.021036 | 0.181 | y = 7.3588x + 0.03 |
| | 0.042072 | 0.347 | R² = 0.9993 |
| | 0.063108 | 0.491 | |
| | 0.084144 | 0.649 | |

Test Results of Sample Products

| | Total sugar (calculated |
|---|---|
| Sample No. | on the basis of glucose, %) |
| 1 | 90 |
| 2 | 92 |
| 3 | 88 |
| 4 | 85 |
| 5 | 95 |

Sample 1 is the material from Example 8 (described below).

Sample 2 is the material from Example 9 (described below).

Sample 3 is the material from Example 10 (described below).

Sample 4 is the material from Example 11 (described below).

Sample 5 is the material from Example 12 (described below).

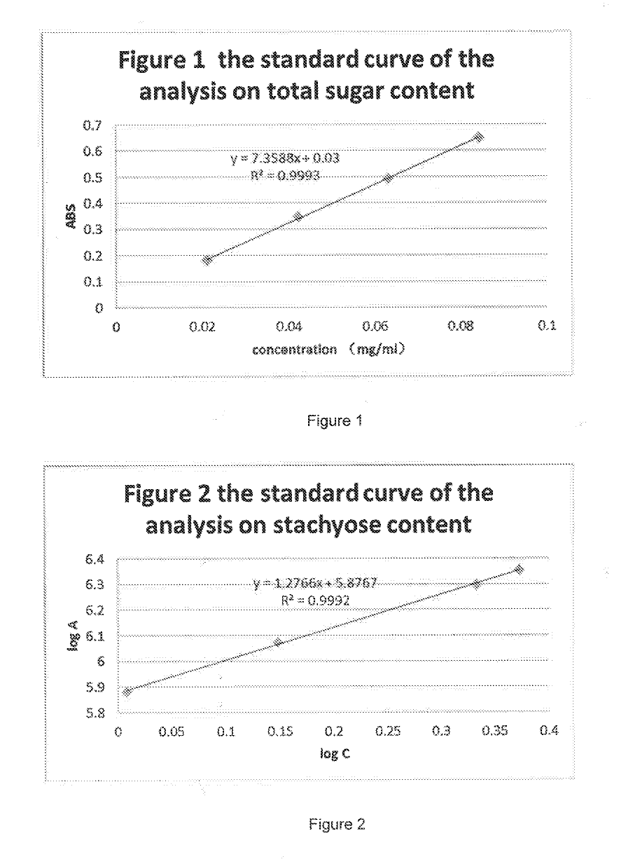

FIG. 1 depicts the standard curve of the analysis on total sugar content in Example 1.

1.3 Conclusion

Total sugar content of the composition obtained from this method is between 85-95%. (Glucose only)

### Example 2 Analysis on Stachyose Content

2.1 Instrument Settings

(Agilent 1100GC-Sedex75 Evaporative Light Scattering Detector)

Chromatographic column: SHISEIDO Capcell Pak NH₂ column (150 mm×4.6 mm. 5 μm), specification of column: GQAB01862

Flow velocity: 1 mL/min

Sample size: 10 μL

Mobile phase: acetonitrile: H₂O=60:40

Operating time: 25 min

Evaporative Light Scattering Detector. 40° C. 3.5 Bar

2.2 Reference Substance Solution

Reference substance (batch No.: SST20160929, content: 89% from Xi'An App-Chem Bio(Tech) Co., Ltd, China) 25 mg, 37.5 mg, 58 mg, 65 mg respectively were added into 25 ml volumetric flasks and brought to volume by addition of water with ultrasonication, followed by filtration with a membrane of a porosity of 0.22 μm.

2.3 Sample Product Solution

50 mg of a sample was placed into 25 ml volumetric flask and brought to volume by addition of water with ultrasonication, and filtered through a membrane of having a porosity of 0.22 μm.

2.4 Results

Formula of Standard Curve:

log A=1.2766 log C+5.8767 (R²=0.9992)

| Sample No. | content (on a dry basis, %) |
|---|---|
| 1 | 85 |
| 2 | 90 |
| 3 | 70 |
| 4 | 75 |
| 5 | 94 |

Sample 1 is the material from Example 8 (described below).

Sample 2 is the material from Example 9 (described below).

Sample 3 is the material from Example 10 (described below).

Sample 4 is the material from Example 11 (described below).

Sample 5 is the material from Example 12 (described below).

FIG. 2 depicts the standard curve of the analysis on stachyose content in example 2.

2.5 Conclusion

Stachyose content of the composition obtained by this method is between 70-94%.

### Example 3 Analysis on Reducing Sugar Content

Principle: reducing sugar could react with dinitrosalicylic acid (DNS) which is a oxidation-reduction reaction. It is feasible to measure the content of a reducing sugar by making use of the principle that the product 3-amino-5-nitro salicylic acid obtained in the reaction above will be brownish red when boiled.

3.1 Preparation of the Solution

Test solution A: 300 ml of a 4.5% NaOH solution, 88 ml of a 1.0% DNS solution and 255 g potassium tartrate were dissolved with ultrasonication.

Test solution B: 22 ml of a 10% NaOH solution and 10 g phenol were added to a 100 ml volumetric flask and dissolved with ultrasonication. Water was added to bring to volume. To 69 ml of solution prepared above was added 6.9 g sodium sulfite and dissolved with ultrasonication.

DNS test solution: Test solution B and test solution A were mixed well and stored in a brown bottle. The DNS test solution can be used for one week after mixing.

3.2 Abs Measurement of the Sample Product

150 mg sample product was added to a 50 ml volumetric flask, dissolved with water and brought to volume by addition of water. 1 ml solution above was added to a 10 ml colorimetric tube with plug, followed by the addition of 3 ml DNS test solution to the tube which was mix with ultrasonication. The tube was placed in a boiling water bath for 10 min followed by cooling under running water. The solution was transferred to a 25 ml volumetric flask and brought to volume by addition of water. A blank solution was prepared with the same method as a reference. The Abs of the solution was measured at 540 nm. Each sample was tested twice to provide an average.

3.3 Standard Curve

25 mg glucose was added to a 50 ml volumetric flask, dissolved with water and brought to volume by addition of water. This was used as the reference substance mother liquor. 0.3 ml, 0.5 ml, 1 ml, 1.5 ml reference substance mother liquor was transferred to 10 ml colorimetric tubes with plugs along with 3 ml DNS test solution. The Abs was measured as described in 1.2.

3.4 Results

Standard Curve

| | Concentration | chromogenic | | |
|---|---|---|---|---|
| Reference | of mother | concentration | Absorbancy | Formula of standard |
| substance(mg) | liquor(mg/ml) | (mg/ml) | (Abs) | curve |
| 25.91 | 0.5182 | 0.006218 | 0.084 | y = 16.382x − |
| | | 0.010364 | 0.158 | 0.0139 (R² = 0.9994) |
| | | 0.020728 | 0.331 | limit of |
| | | 0.031092 | 0.492 | quantitation(Abs of |
| | | | | the blank solution: |
| | | | | 0.000Abs − 0.002Abs): |
| | | | | 10*0.002/16.382 = |
| | | | | 1.22 × 10−3 mg/ml |

Sample Product:

| Sample No. | Absorbance (Abs) |
|---|---|
| 1 | 0.002 |
| 2 | 0.002 |
| 3 | 0.003 |
| 4 | 0.001 |
| 5 | 0.002 |
| 6 | 0.001 |

Sample 1 is the material from Example 8 (described below).

Sample 2 is the material from Example 9 (described below).

Sample 3 is the material from Example 10 (described below).

Sample 4 is the material from Example 11 (described below).

Sample 5 is the material from Example 12 (described below).

Sample 6 is the material from Example 13 (described below).

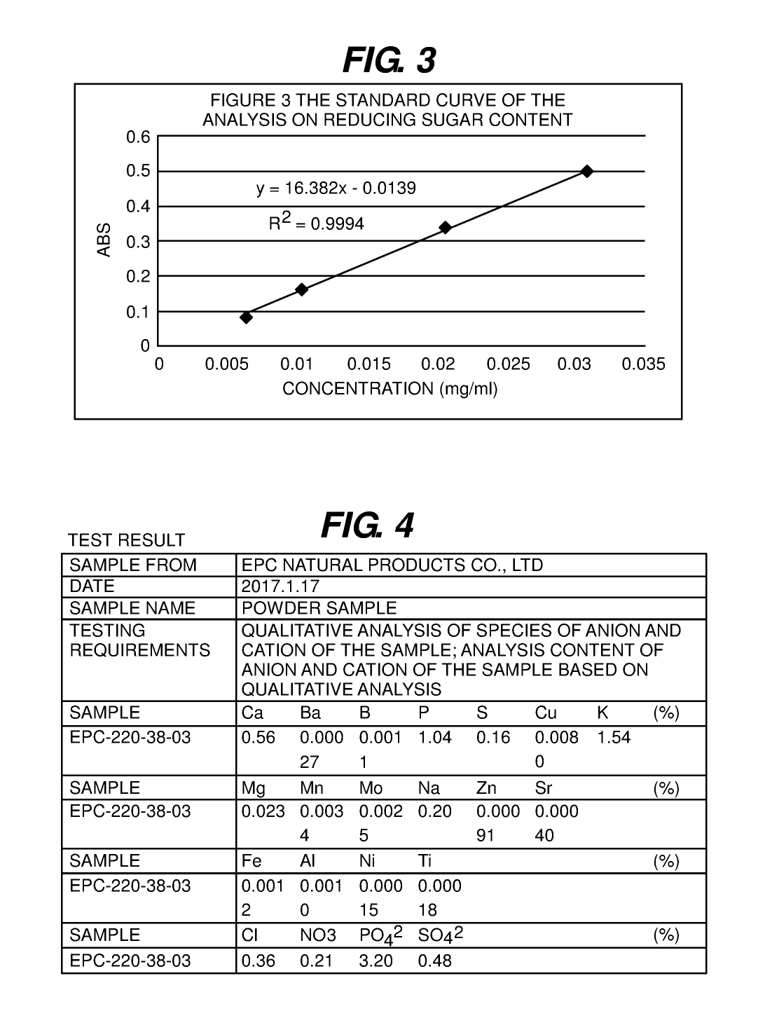

FIG. 3 depicts the standard curve of the analysis on reducing sugar content in Example 3.

The data above shows that Abs of sample product solution is close to the noise of the instrument, that is the reducing sugar is not detected in the sample product. The content of reducing sugar is below 1%(1.22×10⁻³ mg/ml*25 ml/1 ml*50 ml/150 mg*100).

3.5 Conclusion

There is nearly no reducing sugar in the composition obtained with this method.

### Example 4 Analysis of Content of the Salts

Ash content of the sample products are as follows:

| Sample No. | Ash content(%) |
|---|---|
| 1 | 4 |
| 2 | 3 |
| 3 | 10 |
| 4 | 8 |
| 5 | 2 |

Sample 1 is the material from Example 8 (described below).

Sample 2 is the material from Example 9 (described below).

Sample 3 is the material from Example 10 (described below).

Sample 4 is the material from Example 11 (described below).

Sample 5 is the material from Example 12 (described below).

The ion analysis of one batch of sample product shows that, the sample product comprise of:

| | |
|---|---|
| K⁺ | 1.54% |
| Ca²⁺ | 0.56% |
| Cl⁻ | 0.36% |
| NO₃⁻ | 0.21% |
| PO₄³⁻ | 3.20% |
| SO₄²⁻ | 0.48% |

FIG. 4 depicts the ion content in Example 4.

Conclusion:

The ash content of the composition obtained with this method is between 2-10% wt, in which the main cation is K⁺, while the main anion is PO₄³⁻.

### Example 5 Analysis of Content of Amino Acid

The content of amino acids in 5 batches of sample products was analyzed with ion chromatography. There were several kinds of amino acids in the composition obtained by this method, which are mainly arginine, glutamic acid, threonine, asparaginic acid, lysine, valine and serine, etc.

| | | | | Total | |
|---|---|---|---|---|---|
| | | Glutamic | | amino | |
| Sample | Arginine | acid | Threonine | acid | Content |
| No. | (g/kg) | (g/kg) | (g/kg) | (g/kg) | (%) |
| 1 | 10.88 | 8.52 | 2.27 | 26.84 | 2.68 |
| 2 | 8.65 | 4.84 | 2.74 | 25.97 | 2.60 |
| 3 | 14.41 | 12.82 | 2.60 | 38.48 | 3.85 |

Sample 1 is the material from Example 8 (described below).

Sample 2 is the material from Example 9 (described below).

Sample 3 is the material from Example 10 (described below).

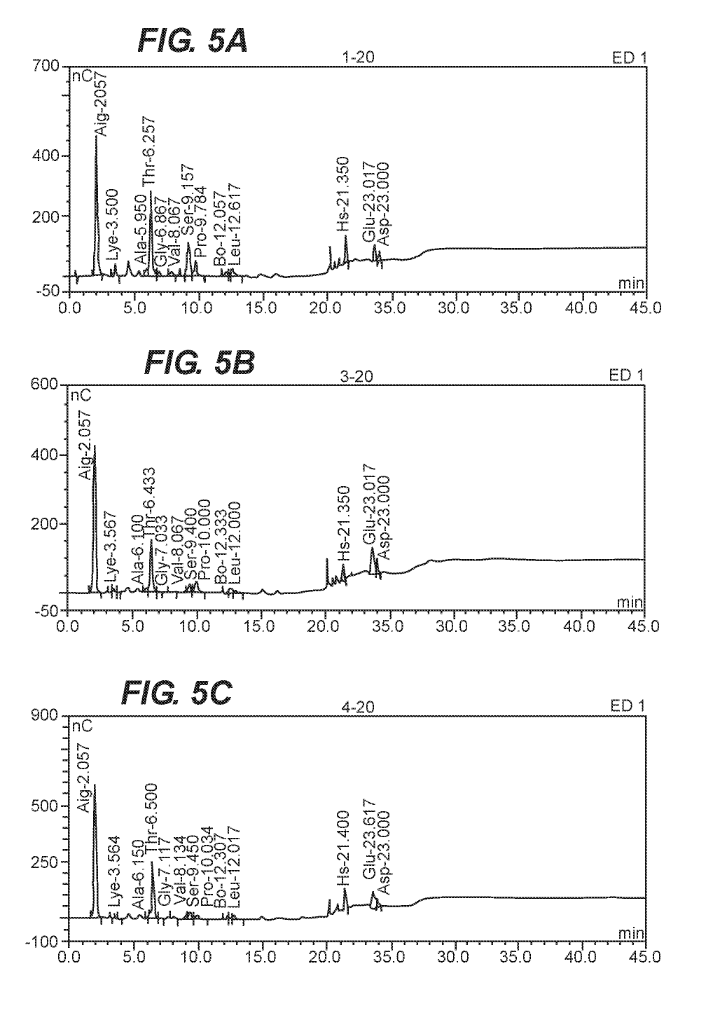

FIGS. 5A, 5B and 5C depict the amino acid content analysis in Example 5.

Conclusion: The content of amino acid in the compositions obtained from this method is between 2-5% wt.

### Example 6 Analysis of Content of Composition Arousing Odor

The following is for a comparison of smell of aqueous solutions by methods noted below. Use of activated carbon was employed to process both of the aqueous solutions in order to absorb volatile substances causing odor. The volatile substances were analyzed with GC-MS qualitatively.

Normal Method Preparing Sample Product (Method A):

500 g Chinese Artichoke and 200 g deionized water were mixed and squeezed at ambient temperature for a period of time between about 10 minutes to about 2 hours and a grey thick liquid (juice) was obtained. The thick liquid was filtered (filter paper, cloth or by centrifugation) to obtain a first filtrate and residue. The residue was mixed with 200 g deionized water and squeezed, as above, and a second grey thick liquid is obtained. The thick liquid was filtered, as above, to obtain a second filtrate. The first and second filtrates were combined and heated to about 45° C. and then Ca(OH)₂ powder was added to the filtrates to adjust the pH to about 9-10 and the solution was allowed to stand for 2 h at approximately 45° C. The solution was cooled to room temperature and any precipitate was removed by filtration. Phosphoric acid was added to the filtrate at room temperature until the pH was adjusted to about 4-5, and the solution was allowed to stand for 1 h at ambient temperature. The precipitate was filtered (filter paper, cloth or centrifuged) and the filtrate was decolorized with activated carbon at about 0.5% to about 2% w/v activated carbon to the total volume of filtrate. The carbon treated solution was stirred for about 30 to about 60 minutes at ambient temperature before the activated carbon was filtered from the solution (filter paper, cloth or by centrifugation) and a brown and clear filtrate was obtained. The solution was spray-dried and an off-white powder was obtained (Method A sample). Some aldehydic material(s) remained in Method A sample as the sample retained some odor. Spray drying conditions: L-117 type lab sprayer available from Laiheng Lab-Equipment, China at a temperature of the inlet at about 110° C. to about 220° C. with a pressure of from about 5 to about 10 bar(s).

Throughout the following examples described herein, the spray drying conditions are as above: L-117 type lab sprayer available from Laiheng Lab-Equipment, China at a temperature of the inlet at about 110° C. to about 220° C. with a pressure of from about 5 to about 10 bar(s).

Method Preparing Sample Product Eliminating Odor (Method B):

The Method A sample obtained above was mixed with water to get a 10% (w/v) solution. Elimination of odor from the Method A sample with activated carbon by treating the solution with a ratio of carbon to solution from about 1 to about 2% weight/volume (w/v) with stirring for approximately 30 minutes to about 60 minutes at a temperature range from about 20° C. to about 50° C. Alternatively, the aqueous solution could be treated with vacuum evaporation or by steam distillation to obtain an enhanced purified product.

When the sample was treated with activated carbon, the solution was filtered (as noted above) and the solution was concentrated by spray-drying to provide a powder noted as Method B sample.

The two sample products obtained from Method A and Method B were treated with deionized water to obtain a 25% (w/v) aqueous solution. The two solutions were treated with activated carbon (0.5% to about 2% w/v) for approximately 1 h at ambient temperature, filtered (filter paper or cloth), and the activated carbon powder was dried at room temperature.

Testing of the two samples on activated carbon with GC-FIT provided a graph that demonstrates the two samples are totally different. (See FIG. 9)

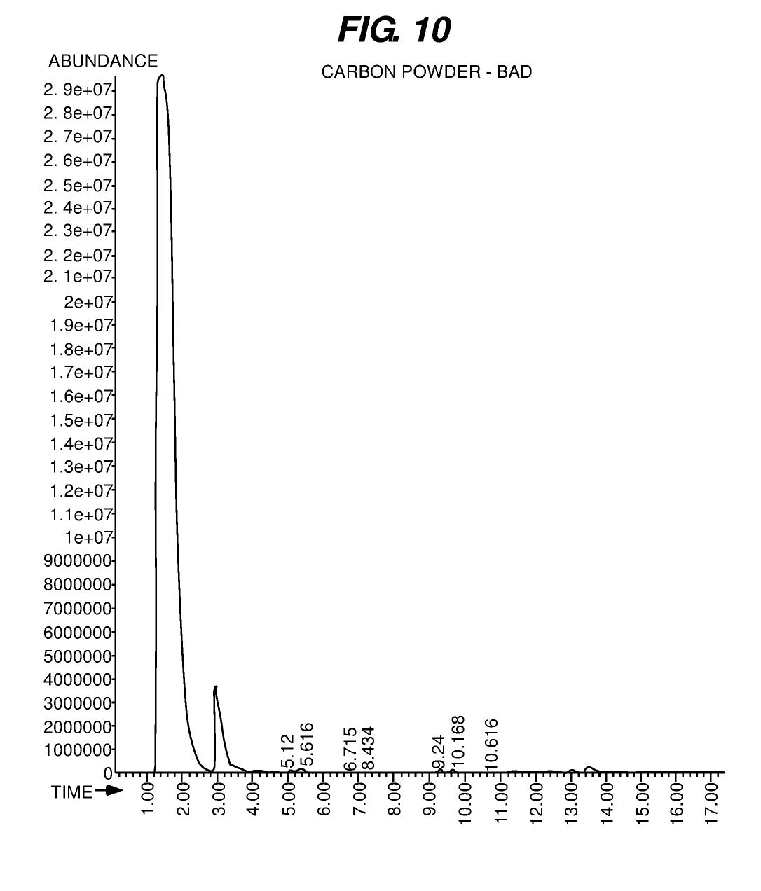

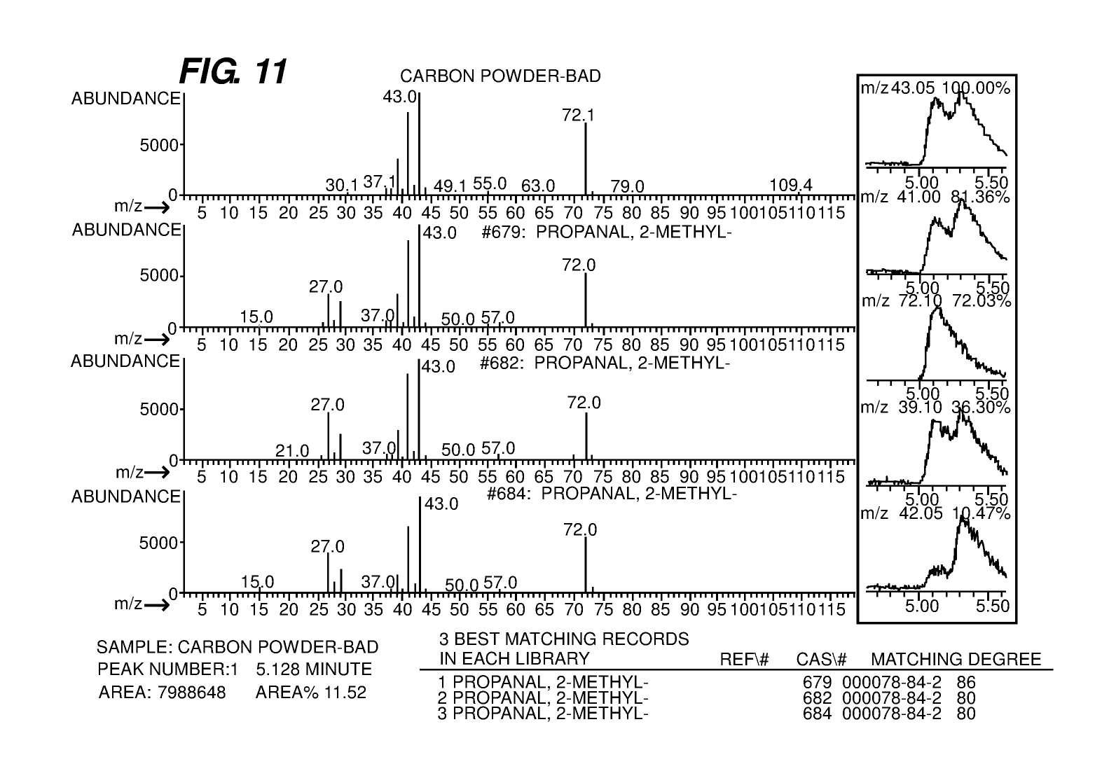

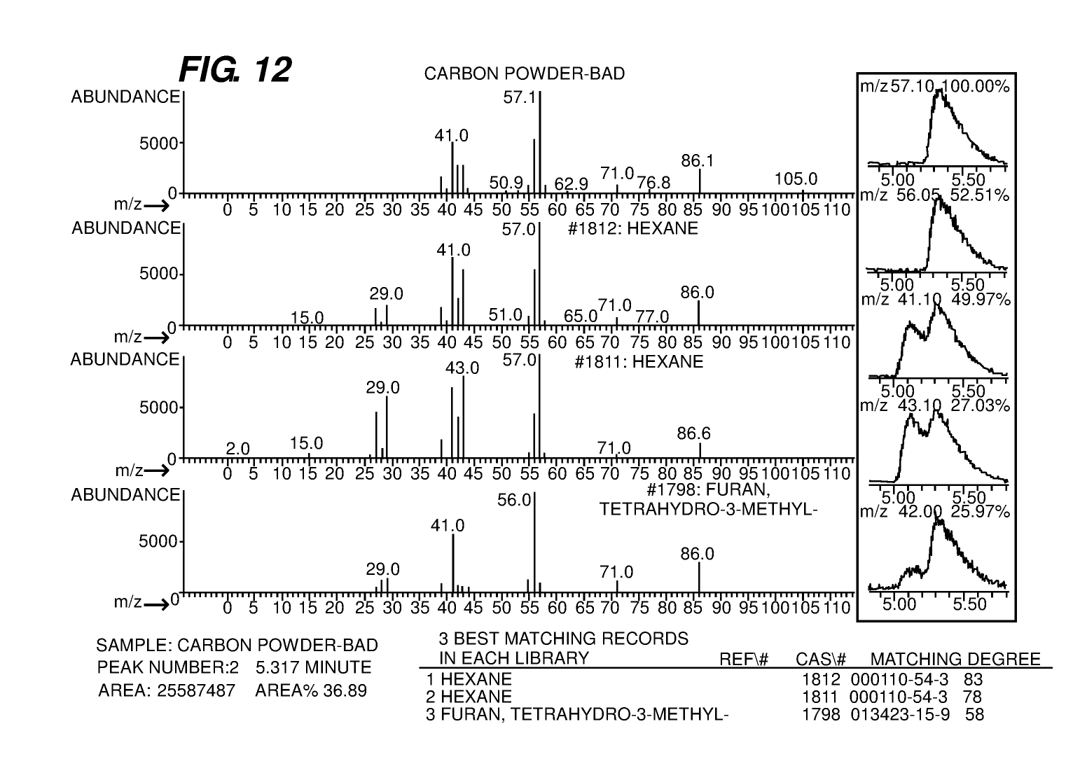

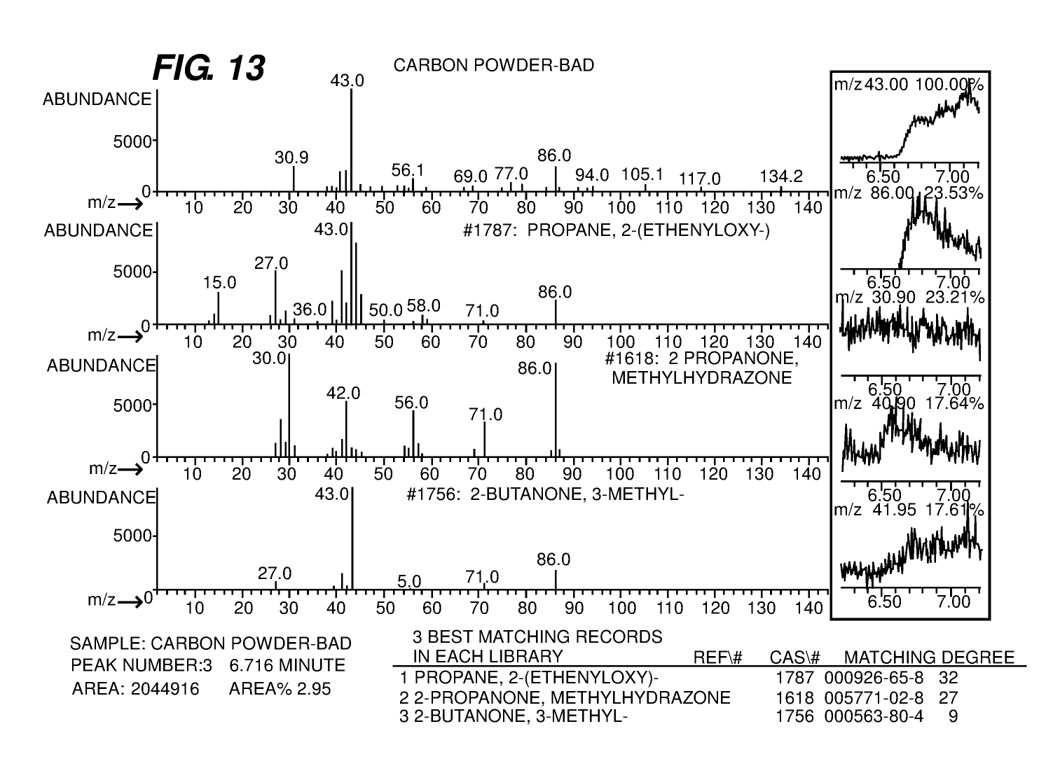

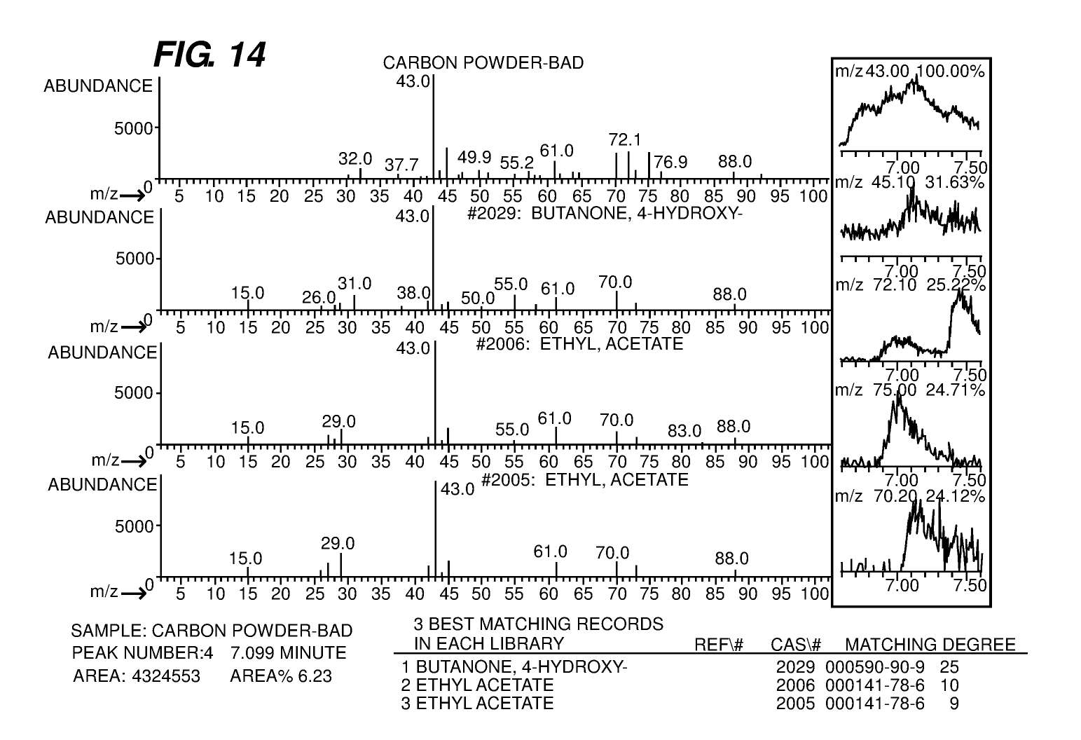

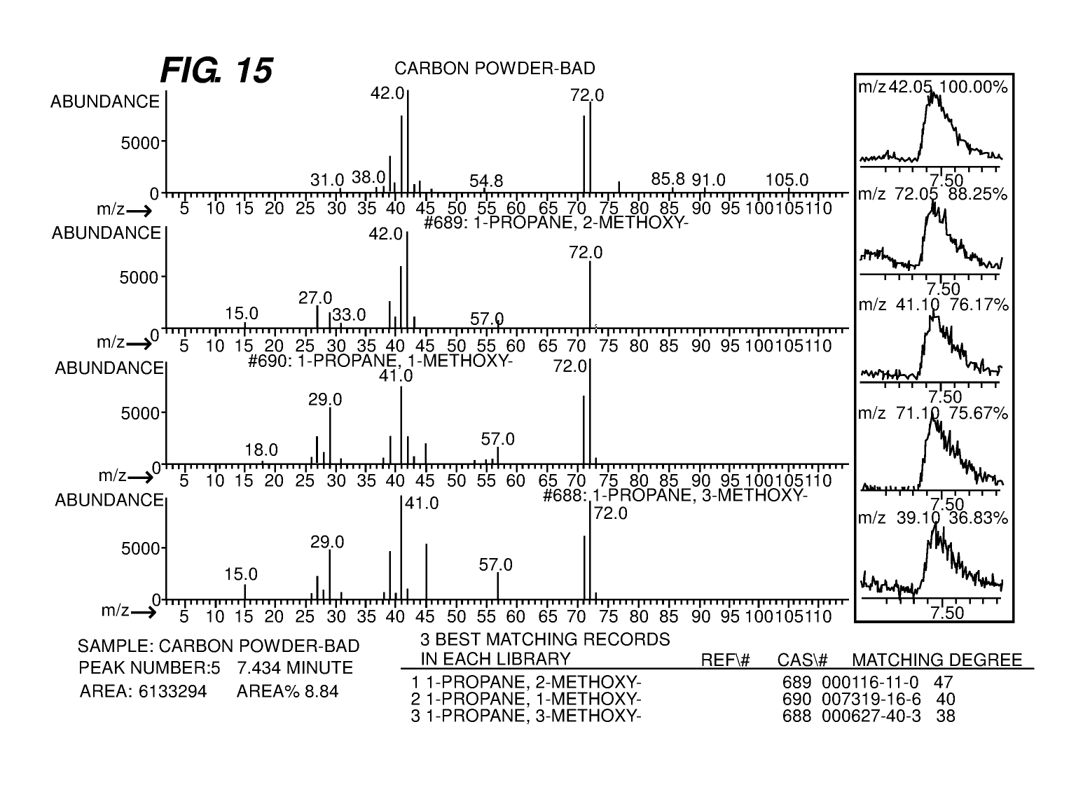

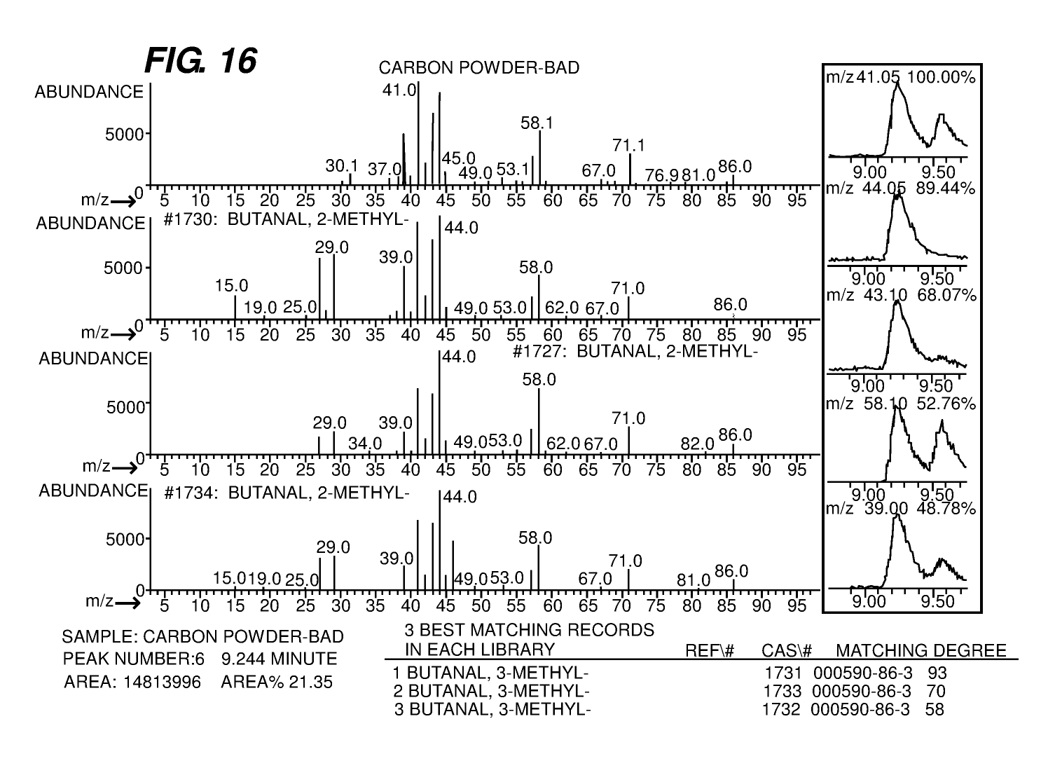

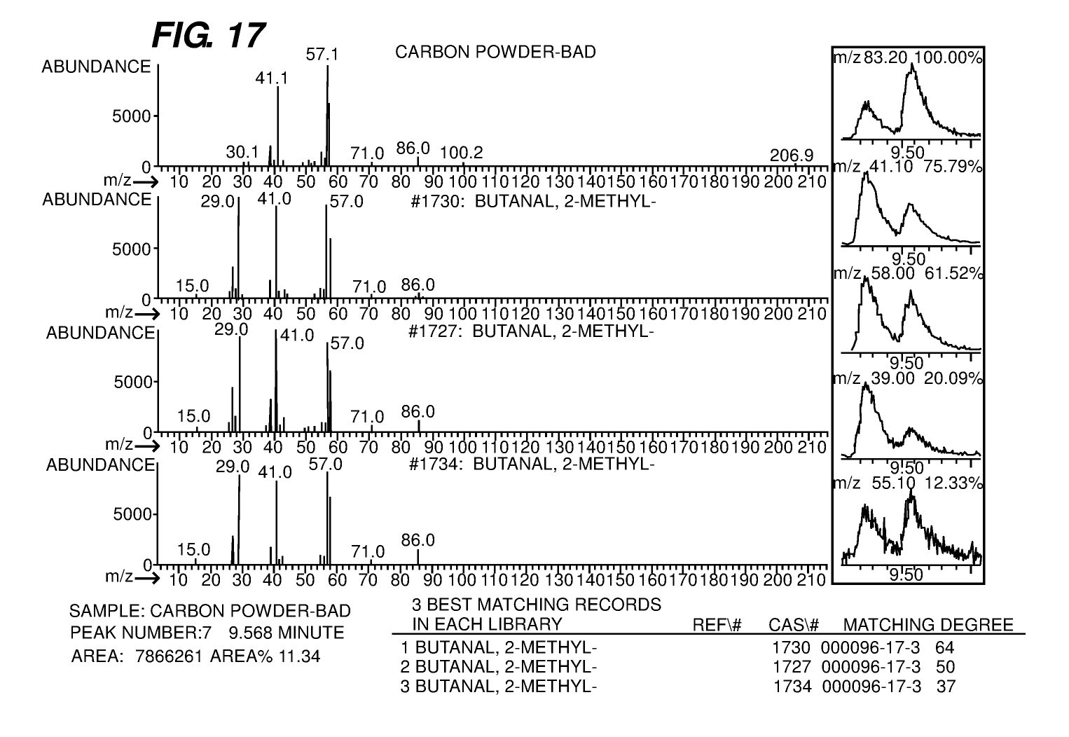

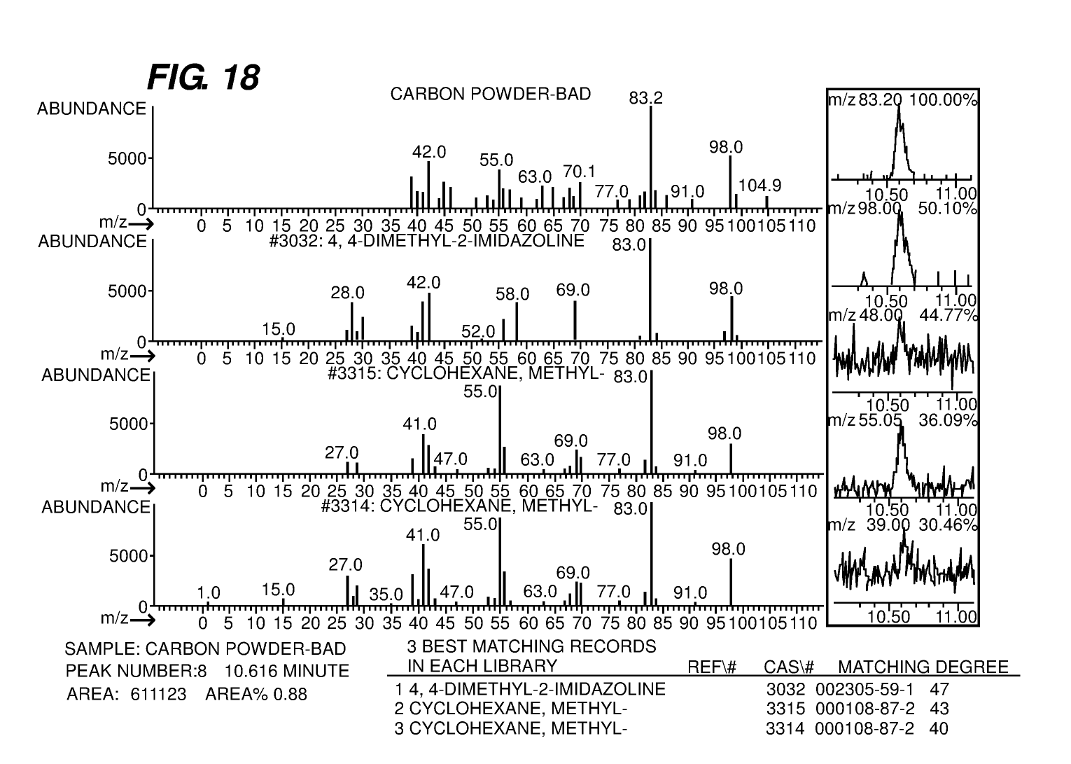

Analysis of the two samples on activated carbon with GC-MS demonstrated that the volatile components were different in the samples. FIG. 10 depicts the GCMS-carbon powder graph of the reference substances prepared in Example 6. FIGS. 11-18 show that there are isobutyl aldehyde, isovaleraldehyde, 2-methyl butylaldehyde in the activated carbon powder in the Method A sample product with odor, while these components were not present in the activated carbon powder processed Method B sample product noted as without odor. Therefore, it was determined that the aldehydes are the substances causing odor in the composition.

Quantitative analysis of stachyose products was performed for the aldehydes above.

6.1 Reference Substance

6.1.1 Information of Reference Substance

isobutyl aldehyde: Aladdin, batch No.: 1507011, content %≥99%

isovaleraldehyde: Aladdin, batch No.: E1618040, content %≥99%

2-methyl butylaldehyde: DuYi, batch No.: CCHM700694-1604, content %≥99.2%

6.1.2 Reference Substance Solution

Add isobutyl aldehyde, isovaleraldehyde and 2-methyl butylaldehyde 100 mg respectively into a 50 ml volumetric flask and bring to volume by addition of DMF. Add 5 ml solution above into a 100 ml volumetric flask and bring to volume by addition of DMF to obtain the reference substance mother liquid.

Reference substance solution 1: add 1 ml reference substance mother liquid into a 250 ml volumetric flask and bring to volume by addition of water. Add 2 ml solution 1 to a headspace bottle and analyze it.

Reference substance solution 2: add 1 ml reference substance mother liquid into a 100 ml volumetric flask and bring to volume by addition of water. Add 2 ml solution 2 to a headspace bottle and analyze it.

Reference substance solution 3: add 1 ml reference substance mother liquid into a 50 ml volumetric flask and bring to volume by addition of water. Add 2 ml solution 3 to a headspace bottle and analyze it.

Reference substance solution 4: add 1 ml reference substance mother liquid into a 25 ml volumetric flask and bring to volume by addition of water. Add 2 ml solution 4 to a headspace bottle and analyze it.

Instrument: Agilent 6890N

Inlet temperature: 180° C.

Column: Agilent125-1334 DB-624 Capillary 30.0 m×530 um×3.00 um nominal

Column temperature: 40° C., hold 5 min, 5° C./min rise to 60° C., 50° C./min rise to 180° C., hold 2 min

Detector temperature (FID): 250° C.

Air flow rate: 400 mL/min

Hydrogen flow rate: 45 mL/min

Vial Temp: 80° C.

Loop Temp: 90° C.

TR. LINE Temp: 100° C.

6.2 Sample Product

1 g sample product was added to a headspace bottle, followed by 2 ml water. The bottle was sealed and the sample was dissolved with ultrasonication and analyzed against the standards.

6.3 Results

6.3.1 Standard Curve

| | | |
|---|---|---|
| isobutyl aldehyde | Y = 23474x + 0.688 | (R2 = 0.9994) |
| isovaleraldehyde | Y = 21397x + 0.3664 | (R2 = 0.9995) |
| 2-methyl butylaldehyde | Y = 16822x + 0.243 | (R2 = 0.9999) |

6.3.2 Test Results of Sample Product

| | content (ppm) | | |
|---|---|---|---|
| | 2-methyl | | isobutyl |
| Sample No. | butylaldehyde | isovaleraldehyde | aldehyde |
| 4 | 0.48 | 0.42 | 0.23 |
| 5 | 0.14 | 0.14 | 0.06 |
| 6 | 0.60 | 0.44 | 0.23 |
| reference sample | 4.95 | 2.38 | 2.23 |
| 1 made by EPC | | | |
| Xian Tianmei 1 | 35.29 | 10.68 | 12.72 |
| Xian Tianmei 2 | 5.41 | 2.60 | 2.30 |
| Xian Tianmei 3 | 2.89 | 1.61 | 1.09 |
| Xian Tianmei 4 | 5.15 | 2.75 | 2.02 |
| Xian Tianmei 5 | 6.24 | 3.40 | 2.43 |

Xian Taianmei samples 1, 2, 3, 4 and 5 were purchased from Xi'An App-Chem Bio(Tech) Co., Ltd, China.

Sample 4 is the product of Example 11 noted below.

Sample 5 is the product of Example 12 noted below.

Sample 6 is the product of Example 13 noted below.

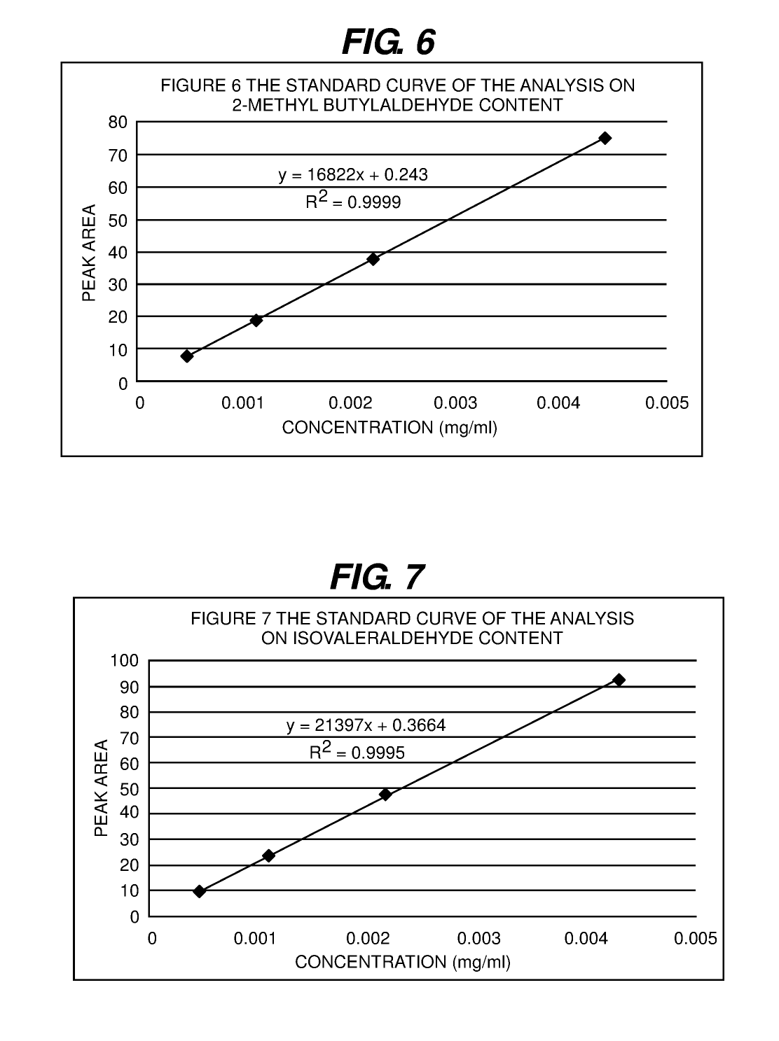

FIG. 6 depicts the standard curve of the analysis on 2-methyl butylaldehyde content in Example 6.

FIG. 7 depicts the standard curve of the analysis on isovaleraldehyde content in Example 6.

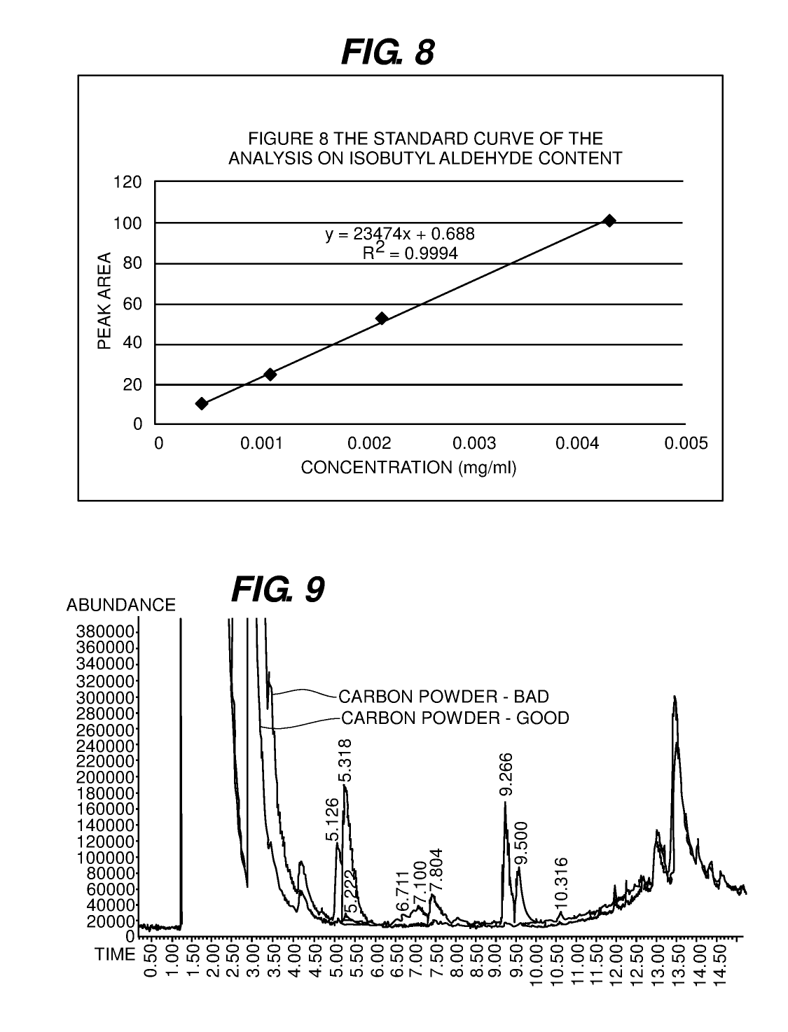

FIG. 8 depicts the standard curve of the analysis on isobutyl aldehyde content in Example 6.

Conclusion: Content of aldehydes in sample products treated to eliminate odor is lower than that of samples without eliminating odor as well as products in the marketplace (Xi'An App-Chem Bio(Tech) Co., Ltd, China).

### Example 7 Comparison of Sensory Evaluation

7.1 Comparison of Sensory Evaluation Between Samples with Odor and Eliminating Odor

7.1.1 Samples

Sample 6 made in Example 13, reference sample 1 made by EPC in example 13 and Xian Tianmei 1 noted in example 6.

7.1.2 Evaluation Method

Samples were dissolved with deionized water to obtain a 5% w/v solution.

A 5 person panel evaluated the smell and texture of the solution. They described the results and kept records.

7.1.3 Results

| | evaluation | | | | | |
|---|---|---|---|---|---|---|
| | smell | | | texture | | |
| | sample No. | | | | | |
| | | Reference | | | Reference | |
| | | sample | Xian | | sample | Xian |
| No. of | | 1 made | Tianmei | | 1 made | Tianmei |
| person | 6 | by EPC | 1 | 6 | by EPC | 1 |
| 1 | Without | odor of | Obvious | Slightly | Slightly | Slightly |
| | odor | carton | odor of | sweet, | sweet, | sweet, |
| | | | carton | umami | puckery | obviously |
| | | | | | | puckery |
| 2 | Without | Obvious | Obvious | Slightly | Slightly | Slightly |
| | odor | odor of | odor of | sweet, | sweet, | sweet, |
| | | carton | carton | umami | puckery | obvious |
| | | | | | | taste of |
| | | | | | | metal |
| 3 | Nearly | Odor of | Obvious | Slightly | Slightly | Slightly |
| | without | carton | odor of | sweet, | sweet, | sweet, |
| | odor | | carton | umami | taste of | taste of |
| | | | | | metal, | metal |
| | | | | | puckery | |
| 4 | Without | Odor of | Obvious | Slightly | Slightly | Slightly |
| | odor | carton | odor of | sweet, | sweet, | sweet, |
| | | | carton | umami | odor | obviously |
| | | | | | | puckery |
| 5 | Without | Obvious | Obvious | Slightly | Slightly | Slightly |
| | odor | odor | odor of | sweet, | sweet, | sweet, |
| | | | carton | umami | taste of | taste of |
| | | | | | metal | metal |

7.1.4 Results

Samples eliminating odor have no odor or taste, with better texture than untreated samples.

7.2 Evaluation of Taste Profile of a Combination of a Polysaccharide with Stachyose and Amino Acids

7.2.1 Materials

Sample 14 was prepared by the process of Example 14

Sample 15 was prepared by the process of Example 15 (90:10)

Sample 16 was prepared by the process of Example 16 (92:8)

Sample 17 was prepared by the process of Example 17 (99.5:0.5)

Sample 18 was prepared by the process of Example 18 (95:5)

7.2.2 Samples Solution

Samples in 7.2.1 were dissolved in deionized water with ultrasound at room temperature and left to sit for 30 min to obtain 10% wt solutions.

7.2.3 Evaluation

Data from a panel of 6 persons. The samples were tested and were scored 0-5 according to increasing saltiness, umami, aftertaste and lingering taste profiles. The results were recorded as the mean value of the results provided by the panel.

| Sample | No. of | Unpleasant | Sweet | |
|---|---|---|---|---|
| No. | person | Aftertaste | lingering | Umami |
| 14 | P1 | 4.00 | 2.50 | 0.00 |
| | P2 | 4.00 | 3.00 | 0.00 |
| | P3 | 4.50 | 3.00 | 0.00 |
| | P4 | 4.00 | 2.00 | 0.00 |
| | P5 | 3.50 | 2.00 | 0.00 |
| | P6 | 3.50 | 2.50 | 0.00 |
| | average | 3.92 | 2.50 | 0.00 |
| 15 | P1 | 0.00 | 0.00 | 5.00 |
| | P2 | 0.00 | 0.00 | 5.00 |
| | P3 | 0.00 | 0.50 | 5.00 |
| | P4 | 0.00 | 0.00 | 5.00 |
| | P5 | 0.00 | 0.00 | 5.00 |
| | P6 | 0.00 | 0.00 | 5.00 |
| | average | 0.00 | 0.08 | 5.00 |
| 16 | P1 | 0.00 | 0.50 | 4.00 |
| | P2 | 0.50 | 0.00 | 4.50 |
| | P3 | 0.00 | 0.50 | 4.00 |
| | P4 | 0.50 | 0.50 | 4.50 |
| | P5 | 0.50 | 0.00 | 4.50 |
| | P6 | 0.50 | 0.00 | 4.50 |
| | average | 0.33 | 0.25 | 4.33 |
| 17 | P1 | 4.00 | 2.50 | 0.50 |
| | P2 | 3.50 | 2.50 | 0.50 |
| | P3 | 4.00 | 2.50 | 1.00 |
| | P4 | 4.00 | 2.00 | 0.50 |
| | P5 | 3.50 | 2.00 | 1.00 |
| | P6 | 3.50 | 2.50 | 0.50 |
| | average | 3.75 | 2.33 | 0.67 |
| 18 | P1 | 0.50 | 1.00 | 3.00 |
| | P2 | 0.50 | 0.50 | 3.50 |
| | P3 | 0.50 | 1.00 | 3.00 |
| | P4 | 1.00 | 0.50 | 3.00 |
| | P5 | 1.00 | 0.00 | 3.50 |
| | P6 | 0.50 | 0.50 | 4.00 |
| | average | 0.67 | 0.58 | 3.33 |

7.2.4 Results

| Sample | Unpleasant | Sweet | |
|---|---|---|---|
| No. | Aftertaste | lingering | Umami |
| 14 | 3.92 | 2.50 | 0.00 |
| 15 | 0.00 | 0.08 | 5.00 |
| 16 | 0.33 | 0.25 | 4.33 |
| 17 | 3.75 | 2.33 | 0.67 |
| 18 | 0.67 | 0.58 | 3.33 |

The combination of polysaccharide and an amino acid improved the taste of the polysaccharide, by elimination of the unpleasant aftertaste, shorting sweet lingering, and provided the product with taste of umami. Shorting is meant to indicate that the duration of the sweet aftertaste is reduced.

7.3 Evaluation of Taste Profile on Combination of a Poly Saccharide with Stachyose and Salts.

7.3.1 Materials

Sample 14 was prepared by the process of Example 14

Sample 19 was prepared by the process of Example 19 (85:15)

Sample 20 was prepared by the process of Example 20 (90:10)

Sample 21 was prepared by the process of Example 21 (99.5:0.5)

Sample 22 was prepared by the process of Example 22 (95:5)

7.3.2 Samples Solution

Samples in 7.3.1 were dissolved in deionized water with ultrasound at room temperature and left to sit for 30 min to obtain a 10% wt solution.

7.3.3 Evaluation

Data from a panel of 6 persons. The samples were tested and were scored 0-5 according to the increasing saltiness, umami, aftertaste and lingering taste profiles. The results were recorded as the mean value of the results provided by the panel.

| Sample | No. of | Unpleasant | Sweet | |
|---|---|---|---|---|
| No. | person | Aftertaste | lingering | Salty |
| 14 | P1 | 4.00 | 3.00 | 0.00 |
| | P2 | 4.00 | 2.00 | 0.00 |
| | P3 | 3.50 | 3.00 | 0.00 |
| | P4 | 3.50 | 2.50 | 0.00 |
| | P5 | 4.00 | 3.00 | 0.00 |
| | P6 | 3.50 | 3.00 | 0.00 |
| | average | 3.75 | 2.75 | 0.00 |
| 19 | P1 | 0.00 | 0.00 | 5.00 |
| | P2 | 0.00 | 0.00 | 5.00 |
| | P3 | 0.00 | 0.00 | 5.00 |
| | P4 | 0.00 | 0.00 | 5.00 |
| | P5 | 0.00 | 0.00 | 5.00 |
| | P6 | 0.00 | 0.00 | 5.00 |
| | average | 0.00 | 0.00 | 5.00 |
| 20 | P1 | 0.00 | 0.00 | 5.00 |
| | P2 | 0.00 | 0.50 | 5.00 |
| | P3 | 0.00 | 0.50 | 5.00 |
| | P4 | 0.00 | 0.00 | 5.00 |
| | P5 | 0.00 | 0.00 | 4.50 |
| | P6 | 0.00 | 0.50 | 5.00 |
| | average | 0.00 | 0.25 | 4.92 |
| 21 | P1 | 3.50 | 3.00 | 0.00 |
| | P2 | 3.00 | 2.50 | 0.50 |
| | P3 | 3.00 | 2.50 | 0.00 |
| | P4 | 3.50 | 3.00 | 0.00 |
| | P5 | 4.00 | 2.50 | 0.50 |
| | P6 | 3.50 | 3.00 | 0.00 |
| | average | 3.42 | 2.75 | 0.17 |
| 22 | P1 | 1.00 | 0.50 | 4.00 |
| | P2 | 0.50 | 1.00 | 3.50 |
| | P3 | 1.00 | 0.50 | 3.50 |
| | P4 | 1.00 | 0.50 | 3.50 |
| | P5 | 0.50 | 0.50 | 4.00 |
| | P6 | 0.50 | 0.50 | 4.00 |
| | average | 0.75 | 0.58 | 3.75 |

7.3.4 Results

| Sample | Unpleasant | Sweet | |
|---|---|---|---|
| No. | Aftertaste | lingering | Salty |
| 14 | 3.75 | 2.75 | 0.00 |
| 19 | 0.00 | 0.00 | 5.00 |
| 20 | 0.00 | 0.25 | 4.92 |
| 21 | 3.42 | 2.75 | 0.17 |
| 22 | 0.75 | 0.58 | 3.75 |

The combination of polysaccharide and an amino acid improved the taste of the polysaccharide by elimination of the unpleasant aftertaste, shorting sweet lingering, and provided a product with the taste of saltiness and umami.

7.4 Evaluation of Taste Profile on Combination of a Polysaccharide with Stachyose, Amino Acids and Salts

7.4.1 Materials

Sample 14 was prepared by the process of Example 14

Sample 23 was prepared by the process of Example 23 (75:10:15)

Sample 24 was prepared by the process of Example 24 (82:8:10)

Sample 25 was prepared by the process of Example 25 (99:0.5:0.5)

Sample 26 was prepared by the process of Example 26 (90:5:5)

7.4.2 Sample Solution

Samples in 7.4.1 were dissolved in deionized water with ultrasound at room temperature and left to sit for 30 min to obtain a 10% wt solution.

7.4.3 Evaluation

Data from a panel of 6 persons. The samples were tested and were scored 0-5 according to the increasing saltiness, umami, aftertaste and lingering taste profiles. The results were recorded as the mean value of the results provided by the panel.

| Sample | No. of | Unpleasant | Sweet | | |
|---|---|---|---|---|---|
| No. | person | Aftertaste | lingering | Salty | Umami |
| 14 | P1 | 4 | 2.5 | 0 | 0 |
| | P2 | 4 | 3.5 | 0 | 0 |
| | P3 | 4.5 | 3 | 0 | 0 |
| | P4 | 4 | 3 | 0 | 0 |
| | P5 | 4 | 2.5 | 0 | 0 |
| | P6 | 4 | 2.5 | 0 | 0 |
| | average | 4.08 | 2.83 | 0.00 | 0.00 |
| 23 | P1 | 0 | 0 | 5 | 5 |
| | P2 | 0 | 0 | 5 | 5 |
| | P3 | 0 | 0 | 5 | 5 |
| | P4 | 0 | 0 | 5 | 5 |
| | P5 | 0 | 0 | 5 | 5 |
| | P6 | 0 | 0 | 5 | 5 |
| | average | 0.00 | 0.00 | 5.00 | 5.00 |
| 24 | P1 | 0 | 0 | 5 | 5 |
| | P2 | 0 | 0 | 5 | 5 |
| | P3 | 0 | 0.5 | 5 | 4.5 |
| | P4 | 0.5 | 0 | 5 | 5 |
| | P5 | 0 | 0.5 | 5 | 5 |
| | P6 | 0 | 0 | 5 | 5 |
| | average | 0.08 | 0.17 | 5.00 | 4.92 |
| 25 | P1 | 4 | 3 | 0 | 0.5 |
| | P2 | 4 | 3 | 0.5 | 1.5 |
| | P3 | 4 | 2.5 | 0.5 | 1 |
| | P4 | 4 | 3 | 0.5 | 0.5 |
| | P5 | 3.5 | 2.5 | 0.5 | 1 |
| | P6 | 4 | 3 | 0 | 0.5 |
| | average | 3.92 | 2.83 | 0.33 | 0.83 |
| 26 | P1 | 0.5 | 1 | 4 | 4 |
| | P2 | 0.5 | 0.5 | 3.5 | 3.5 |
| | P3 | 0.5 | 1 | 3.5 | 3 |
| | P4 | 1 | 0.5 | 4 | 3 |
| | P5 | 1 | 0 | 4 | 3 |
| | P6 | 0.5 | 0.5 | 4 | 4 |
| | average | 0.67 | 0.58 | 3.83 | 3.42 |

7.4.4 Results

| Sample | Unpleasant | Sweet | | |
|---|---|---|---|---|
| No. | Aftertaste | lingering | Salty | Umami |
| 14 | 4.08 | 2.83 | 0.00 | 0.00 |
| 23 | 0.00 | 0.00 | 5.00 | 5.00 |
| 24 | 0.08 | 0.17 | 5.00 | 4.92 |
| 25 | 3.92 | 2.83 | 0.33 | 0.83 |
| 26 | 0.67 | 0.58 | 3.83 | 3.42 |

The combination of polysaccharide, an amino acid and a salt improved the taste of polysaccharide by elimination of the unpleasant aftertaste, shorting sweet lingering, and provided the product with a taste of saltiness and umami.

### Example 8 Preparation of Sample Product 1 (Ratio of Starting Materials is 4:1)

400 g Chinese artichoke and 100 g deionized water were mixed at ambient temperature and the mixture was squeezed, crushed, ground, smashed for about 10 minutes to about 2 hours to provide a grey thick liquid (a juice). The thick liquid was filtered to provide a first filtrate and residue. The residue was mixed and squeezed with 100 g deionized water as described above to provide a second grey thick liquid. The thick liquid was filtered to obtain a second filtrate. The first and second filtrates were combined and heated to about 45° C., to which enough Ca(OH)₂ powder was added to adjust the pH to 9-10. The basified solution was allowed to stand for 2 h at about 45° C. The solution was then cooled to ambient temperature prior to filtration. The precipitate was filtered and phosphoric acid was added to the filtrate at room temperature until the pH of the filtrate was about 4-5. The filtrate was allowed to stand for 1 h at room temperature. The resulting precipitate was filtered and decolorized with activated carbon for about 30 minutes (about 2% w/v carbon/filtrate). The activated carbon was filtered from the solution and a brown and clear filtrate was obtained. The filtrate was spray dried to afford an off-white powder. The powder was dissolved in deionized water to obtain a 10% (w/v) solution and treated with activated carbon at a ratio of carbon to solution from about 1 to about 2% weight/volume (w/v) for about 0.5 h at room temperature. The activated carbon was removed by filtration and the filtrate was spray dried to afford 14.2 g of a white powder without odor.

### Example 9 Preparation of Sample Product 2 (Ratio of Starting Materials is 2:1)

500 g Chinese artichoke and 250 g deionized water were mixed at ambient temperature and squeezed, crushed, ground, smashed for about 10 minutes to about 2 hours to provide a grey thick liquid (a juice). The grey thick liquid was filtered to provide a first filtrate and a residue. The residue was mixed with 250 g deionized water at ambient temperature and squeezed as described above to provide a second thick grey liquid. The thick grey liquid was filtered to obtain a second filtrate. The first and second filtrates were combined and heated to about 45° C. and treated with enough Ca(OH)₂ powder to adjust the pH to 10-10.5 and allowed to stand for 3 h at about 45° C. This solution was then cooled to room temperature prior to filtration. The precipitate was filtered and hydrochloric acid was added to the filtrate at room temperature until the pH was about 5-6 and allowed to stand at ambient temperature for about 2 h. The precipitate was filtered and the filtrate was decolorized with activated carbon for approximately 30 minutes (2% w/v, carbon/filtrate). The activated carbon was removed by filtration and a brown and clear filtrate was obtained which was spray-dried to provide an off-white powder. The powder was dissolved in deionized water to provide a 10% (w/v) solution which was treated with activated carbon for approximately 1 h at ambient temperature (about 2% w/v, carbon/solution). The activated carbon was removed by filtration and the filtrate was spray dried to provide 17.2 g of a white powder without odor.

### Example 10 Preparation of Sample Product 3 (Ratio of Starting Materials is 3:1)

300 g Chinese artichoke and 100 g deionized water were mixed and squeezed, smashed, crushed, ground at ambient temperature from about 10 minutes to about 2 hours to provide a grey thick liquid (a juice). The grey thick liquid was filtered to provide a first filtrate and residue. The residue was mixed with 100 g deionized water and squeezed at room temperature as described above to provide a second grey thick liquid. The grey thick liquid was filtered to provide a second filtrate. The first and second filtrates were combined and heated to about 45° C. followed by addition of NaOH powder to adjust the pH of the filtrate to about 10.5-11 and allowed to stand for 4 h at about 45° C. This solution was cooled to ambient temperature prior to filtration. The precipitate was filtered and acetic acid was added to the filtrate at room temperature until the pH was about 6-7 and allowed to stand for approximately 1.5 h at ambient temperature. The precipitate was filtered and the filtrate was treated with approximately 2% activated carbon at room temperature (2% w/v, carbon/filtrate). The activated carbon was removed by filtration and a brown and clear filtrate was obtained which was spray dried to provide an off white powder. The powder was dissolved in deionized water to provide a 10% (w/v) solution. The solution was treated with vacuum evaporation, under 60° C., vacuum degree 0.08˜-0.09 MPa, where half the volume of the water was removed. The concentrated solution was spray dried to provide 11.4 g white powder without odor.

### Example 11 Preparation of Sample Product 4 (Ratio of Starting Materials is 1:1)

350 g Chinese artichoke and 350 g deionized water were mixed and squeezed, smashed, crushed, ground at ambient temperature for about 10 minutes to about 2 hours to provide a grey thick liquid. The grey thick liquid (a juice) was filtered to provide a first filtrate and a residue. The residue was mixed with 350 g deionized water and squeezed at ambient temperature as described above to provide a second grey thick liquid. The grey thick liquid was filtered to provide a second filtrate. The first and second filtrates were combined and heated to about 45° C. to which KOH powder was added to adjust the pH of the filtrate to about 10-11 and allowed to stand for approximately 3 h at about 45° C. The solution was cooled to ambient temperature prior to filtration. The precipitate was filtered and the filtrate was treated with sulfuric acid at room temperature until the pH was about 6-6.5 and was allowed to stand for approximately 2 h at ambient temperature. The precipitate was filtered and treated with activated carbon. The activated carbon was removed by filtration and a brown and clear filtrate was obtained which was spray dried to provide an off-white powder. The powder was dissolved with deionized water to provide a 20% (w/v) solution which was subjected to steam distillation for 10 min. The distillate was discarded and the residue was passed through a reverse osmosis membrane (25° C., 27.6 bar, membrane area 0.22 m², permeate flux 38(±25%)L/m2×h), followed by concentration of the solution by spray-drying, to afford 13.7 g of a white powder without odor.

### Example 12 Preparation of Sample Product 5 (Ratio of Starting Materials is 0.5:1)

200 g Chinese artichoke and 400 g deionized water were mixed and squeezed, smashed, ground, crushed for about 10 minutes to about 2 hours at ambient temperature to provide a grey thick liquid (a juice). The grey thick liquid was filtered to afford a first filtrate and residue. The residue was mixed and squeezed with 400 g deionized water at ambient temperature as described above to provide a second grey thick liquid which was filtered to provide a second filtrate. The first and second filtrates were combined and heated to about 45° C. to which NH₄OH powder was added to adjust the pH to about 10-10.5 and allowed to stand for about 3 h at about 45° C. The solution was cooled to ambient temperature prior to filtration. The precipitate was filtered and citric acid was added to the filtrate at room temperature until the pH was about 6-6.5 and allowed to stand for about 2 h. The precipitate was filtered and the filtrate was treated with activated carbon at ambient temperature for about 30 minutes (approximately 2% w/v, carbon/filtrate). The activated carbon was removed by filtration to afford a brown and clear filtrate, which was spray dried to afford an off-white powder. The powder was dissolved with deionized water to provide a 10% (w/v) solution which was treated with activated carbon for approximately 3 hours at ambient temperature. The activated carbon was removed by filtration and the filtrate was spray-dried to afford 10.1 g of a white powder without odor.

### Example 13 Preparation of Sample Product 6 and Reference Sample 1 Made by EPC

1097 g Chinese artichoke and 600 g deionized water were mixed and squeezed, smashed, crushed, ground at ambient temperature for about 10 minutes to about 2 hours to provide a grey thick liquid (a juice). The grey thick liquid was filtered to provide a first filtrate and residue. The residue was mixed and squeezed with 600 g deionized water at ambient temperature as described above to afford a grey thick liquid that was filtered to provide a second filtrate. The first and second filtrates were combined and heated to about 45° C. to which was added Ca(OH)₂ powder to adjust the pH to about 10-10.5 and allowed to stand for approximately 3 h at about 45° C. The solution was cooled to ambient temperature prior to filtration. The precipitate was filtered and malic acid was added to the filtrate at room temperature until the pH was about 6-6.5 and allowed to stand for approximately 2 h at ambient temperature. The precipitate was filtered and the filtrate was treated with activated carbon at ambient temperature for about 30 minutes (about 2% w/v, carbon/filtrate). The activated carbon was removed with filtration and a brown and clear filtrate was obtained that was spray-dried to afford an off-white powder (Reference Sample 1 made by EPC)

The powder was dissolved with deionized water to obtain a 20% (w/v) solution which was treated by steam distillation for 10 min. The distillate was discarded and the residual solution was passed through a reverse osmosis membrane (25° C., 27.6 bar, membrane area 0.22 m², permeate flux 38(±25%)L/m2×h) followed by concentration of the solution by spray-drying to afford 60.1 g of a white powder without odor. (Sample Product 6)

### Example 14 Preparation of a Polysaccharide Composition

400 g Chinese artichoke and 100 g deionized water were mixed and squeezed, smashed, crushed, ground at ambient temperature for about 10 minutes to about 2 hours to afford a grey thick liquid (juice). The grey thick liquid was filtered to provide a first filtrate and a residue. The residue was mixed with 600 g deionized water and squeezed at ambient temperature as described above to provide a grey thick liquid that was filtered to provide a second filtrate. The first and second filtrates were combined and the combined filtrate was treated with an ultrafiltration membrane of 5000 D. NaOH was added to the ultrafiltered liquid until the pH was between about 9-10 followed by about 2 h static settlement at a temperature at or below about 45° C. The solution was cooled to ambient temperature prior to filtration. The solution was filtered and the filtrate was treated with acetic acid until the pH was between about 6-7, followed by 1 h static settlement at room temperature. The solution was filtered and the filtrate was treated with activated carbon for about 3 hours at ambient temperature (about 2% w/v, carbon/solution). The carbon was removed by filtration and the filtrate was concentrated by spray drying to afford a powder. The powder was dissolved in 90% ethanol in a ratio of 3:1 ethanol/powder (v/w) with heating to between about 75° C. and 80° C. to dissolve the solids. The solution is cooled to room temperature and allowed to remain at room temperature for approximately 48 hours and then filtered to obtain crystals. The crystals were washed with 95% ethanol followed by forced air drying or vacuum oven drying to afford 10.1 g of high purity stachyose.

### Example 15 Preparation of a Composition Comprising a Polysaccharide and an Amino Acid

90 g polysaccharide made according to example 14 and 10 g arginine were mixed well to obtain a composition with better flavor.

### Example 16 Preparation of a Composition Comprising a Polysaccharide and an Amino Acid

92 g polysaccharide made according to example 14 and 8 g glutamic acid were mixed well to obtain a composition with better flavor.

### Example 17 Preparation of a Composition Comprising a Polysaccharide and an Amino Acid

99.5 g polysaccharide made according to example 14 and 0.5 g threonine were mixed well to obtain a composition with better flavor.

### Example 18 Preparation of a Composition Comprising a Polysaccharide and an Amino Acid

95 g polysaccharide made according to example 14 and 5 g aspartic acid were mixed well to get a composition with better flavor.

### Example 19 Preparation of a Composition Comprising a Polysaccharide and a Salt

85 g polysaccharide made according to example 14 and 15 g KCl were mixed well to obtain a composition with better flavor.

### Example 20 Preparation of a Composition Comprising a Polysaccharide and a Salt

90 g polysaccharide made according to example 14 and 10 g CaSO₄ were mixed well to obtain a composition with better flavor.

### Example 21 Preparation of a Composition Comprising a Polysaccharide and a Salt

99.5 g polysaccharide made according to example 14 and 0.5 g Cu(NO₃)₂ were mixed well to obtain a composition with better flavor.

### Example 22 Preparation of a Composition Comprising a Polysaccharide and a Salt

95 g polysaccharide made according to example 14 and 5 g Cu(NO₃)₂ were mixed well to obtain a composition with better flavor.

### Example 23 Preparation of a Composition Comprising a Polysaccharide, an Amino Acid and a Salt

75 g polysaccharide made according to example 14, 10 g lysine and 15 g Na₃PO₄ were mixed well to obtain a composition with better flavor.

### Example 24 Preparation of a Composition Comprising a Polysaccharide, an Amino Acids and a Salt

82 g polysaccharide made according to example 14, 8 g valine and 10 g MgCl₂ were mixed well to obtain a composition with better flavor.

### Example 25 Preparation of a Composition Comprising a Polysaccharide, an Amino Acid and a Salt

99 g polysaccharide made according to example 14, 0.5 g serine and 0.5 g MnSO₄ were mixed well to obtain a composition with better flavor.

### Example 26 Preparation of a Composition Comprising a Polysaccharide, an Amino Acid and a Salt

90 g polysaccharide made according to example 14, 5 g alanine and 5 g MoPO₄ were mixed well to obtain a composition with better flavor.

### Example 27 Preparation of a Polysaccharide Composition without Odor

400 g Chinese artichoke and 100 g deionized water were mixed and squeezed, smashed, crushed, ground for about 10 minutes to about 2 hours at ambient temperature to provide a grey thick liquid. The grey thick liquid was filtered to provide a first filtrate and a residue. The residue was mixed and squeezed with 600 g deionized water at ambient temperature as described above to provide a second grey thick liquid. The second grey thick liquid was filtered to provide a second filtrate. The first and second filtrates were combined and filtered through an ultrafiltration membrane of 5000 D to provide a filtrate. NaOH was added to the filtrate until the pH was between about 9-10, followed by about 2 h static settlement at a temperature below about 45° C. The solution was filtered and acetic acid was added to the filtrate until the pH was between about 6-7, followed by 1 h static settlement at room temperature. The solution was filtered and the filtrate was treated with activated carbon for about 3 hours at ambient temperature (about 2% w/v, carbon/filtrate). The carbon was removed by filtration and the filtrate was concentrated by spray drying to afford a powder. The powder was dissolved in 90% ethanol in a ratio of 3:1 ethanol/powder (v/w) with heating to about 75° C. to about 80° C. to dissolve the solids. The solution was cooled to room temperature and allowed to remain at room temperature for approximately 48 hours, followed by filtration to obtain crystals. The crystals were washed with 95% ethanol followed by forced air drying or vacuum oven drying to afford 10.1 g of high purity stachyose.

The polysaccharide obtained above was dissolved in deionized water to obtain a 10% (w/v) solution and treated with activated carbon for about 3 hours at ambient temperature (about 2% w/v, carbon/solution). The activated carbon was removed by filtration and the filtrate was spray-dried to afford 9.1 g white powder without odor.

### Example 28 Preparation of a Composition Comprising a Polysaccharide and an Amino Acid without Odor

90 g polysaccharide made according to example 14 and 10 g glycine were mixed well to obtain a composition with better flavor. The mixture was dissolved with deionized water to obtain a 10% (w/v) solution and treated with activated carbon for between about 30 minutes to about 3 h at ambient temperature with a ratio of carbon to solution from about 1 to about 2% weight/volume (w/v). The activated carbon was removed by filtration and the filtrate was spray-dried to afford 100 g white powder without odor.

### Example 29 Preparation of a Composition Comprising a Polysaccharide and an Amino Acid without Odor

92 g polysaccharide made according to example 14 and 8 g proline were mixed well to obtain a composition with better flavor. The powder was dissolved in deionized water to obtain a 10% (w/v) solution and vacuum evaporated, at 60° C., vacuum degree 0.08˜-0.09 MPa, to evaporate half the volume of the water followed by concentrated of the solution by spray-drying to afford 100 g of a white powder without odor.

### Example 30 Preparation of a Composition Comprising a Polysaccharide and an Amino Acid without Odor

99.5 g polysaccharide made according to example 14 and 0.5 g leucine were mixed well to obtain a composition with better flavor. The powder was dissolved in deionized water to obtain a 20% (w/v) solution and subjected to steam distillation for 10 min. The distillate was discarded and the residual solution was passed through a reverse osmosis membrane (25° C., 27.6 bar, membrane area 0.22 m², permeate flux 38(±25%)L/m2×h) which was then concentrated by spray-drying to afford 100 g of a white powder without odor.

### Example 31 Preparation of a Composition Comprising a Polysaccharide an Amino Acid without Odor

95 g polysaccharide made according to example 14 and 5 g histidine were mixed well to obtain a composition with better flavor. The mixture was dissolved in deionized water to obtain a 10% (w/v) solution and treated with activated carbon for 3 h at ambient temperature with stirring with a ratio of carbon to solution from about 1 to about 2% weight/volume (w/v). The activated carbon was removed by filtration and the filtrate was spray dried to afford 100 g of a white powder without odor.

### Example 32 Preparation of a Composition Comprising a Polysaccharide and a Salt without Odor

85 g polysaccharide made according to example 14 was dissolved in deionized water to obtain a 10% (w/v) solution and treated with activated carbon for 3 h at ambient temperature with stirring with a ratio of carbon to solution from about 1 to about 2% weight/volume (w/v). The activated carbon was removed by filtration and the filtrate was spray dried to afford 85 g of a white powder without odor. 85 g of the polysaccharide and 15 g FeSO₄ were mixed well to obtain a composition with better flavor.

### Example 33 Preparation of a Composition Comprising a Polysaccharide and a

90 g polysaccharide made according to example 14 was dissolved in deionized water to obtain a 10% (w/v) solution and subjected to vacuum evaporation, at 60° C., vacuum degree 0.08˜-0.09 MPa, to evaporate half the volume of the water. The concentrate was spray dried to afford 90 g of a white powder without odor. 90 g of the polysaccharide and 10 g FeCl₃ were mixed well to obtain a composition with better flavor.

### Example 34 Preparation of a Composition Comprising a Polysaccharide and a Salt without Odor

99.5 g polysaccharide made according to example 14 was dissolved in deionized water to obtain a 20% (w/v) solution and subjected to steam distillation for 10 min. The distillate was discarded and the residual solution was passed through a reverse osmosis membrane (25° C., 27.6 bar, membrane area 0.22 m², permeate flux 38(±25%)L/m2×h) and the concentrated solution was spray dried to afford 99.5 g of a white powder without odor.

99.5 g polysaccharide prepared above and 0.5 g AlCl₃ were mixed well to obtain a composition with better flavor.

### Example 35 Preparation of a Composition Comprising a Polysaccharide and a Salt without Odor

95 g polysaccharide made according to example 14 was dissolved in deionized water to obtain a 10% (w/v) solution and treated with activated carbon for 3 h at ambient temperature with stirring with a ratio of carbon to solution from about 1 to about 2% weight/volume (w/v). The activated carbon was removed by filtration and the filtrate was spray dried to afford 95 g of a white powder without odor. The 95 g polysaccharide and 5 g NaCl were mixed well to obtain a composition with better flavor.

### Example 36 Preparation of a Devil's Claw Extract

Sample Preparation

300 g of devil's claw powder was added to 3 L of water, and extracted under reflux at 80-90° C. for 2 h. After collecting the first extraction solution by filtration, the filter cake was added to 3 L of water and extracted under reflux at 80-90° C. for 2 h. The second extraction solution was collected by filtration. The combined extraction solutions were cooled to 20-30° C. The impurities of the mixed extraction solution were removed by macroporous adsorption resin. The sample was obtained by concentrating and drying the purification solution with a reduced pressure rotary evaporator. The total weight of the sample was 145 g.

Two additional samples were prepared based on the similar procedure as described above except for the proportion of devil's claw powder to extract solvent.

Detection Method

The sample solution was detected by high performance liquid chromatography under the following conditions:

Flow rate: 1 mL/min;

Injection volume: 10 μl;

Sedex75 ELSD: 3.3 bar, 40° C.;

Column: SHISEIDO CAPCELL PAK NH2 (5 μm, 4.6 mm×150 mm);

Mobile phase: 40:60 of water (A): Acetonitrile (B)

The three prepared samples were analyzed by HPLC under the above conditions. The results were reported as follow in the table below: (all contents were based on the total weight of the sample.

| Samples | Stachyose | Harpagoside |
|---|---|---|
| Number | (wt %) | (wt %) |
| 1 | 65.2 | 0 |
| 2 | 56.7 | 0.2 |
| 3 | 51.2 | 1.1 |

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. An oligosaccharide composition comprising stachyose devoid of odor.

2. An oligosaccharide composition comprising stachyose, wherein one or more odiferous aldehydes are not present.

3. The oligosaccharide composition of claim 2, wherein the one or more odiferous aldehydes that are not present comprise isobutyl aldehyde, isovaleraldehyde, 2-methyl butyl aldehyde or mixtures thereof.

4. The oligosaccharide composition of claim 1, further comprising oligosaccharide melitriose (raffinose) or verbascose or mixtures thereof.

5. The oligosaccharide composition of claim 4, further comprising an amino acid.

6. The oligosaccharide composition of claim 5, wherein the amino acid is one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or mixtures thereof.

7. The oligosaccharide composition of claim 6, wherein the oligosaccharide(s) are present from about 92% to about 99.5% by weight total oligosaccharide(s) with an amino acid content of from about 0.5% to about 8% by weight.

8. The oligosaccharide composition of claim 6, wherein the oligosaccharide(s) are present in about 95% by weight total oligosaccharide(s) with an amino acid content of about 5% by weight.

9. The oligosaccharide composition of claim 6, wherein the stachyose content in the oligosaccharide composition with amino acid(s) is from about 55% to about 94% by weight or from about 70% to about 90% by weight.

10. The oligosaccharide composition of claim 4, further comprising a metal salt.

11. The oligosaccharide composition of claim 4, further comprising one or more prebiotics.

12. The oligosaccharide composition of claim 11, wherein the one or more prebiotic comprises fructooligosaccharides, galactooligosaccharides, arabinose, galactose, inulin, raffinose, mannose, lactulose, mannanoligosaccharides, xylooligosaccharides, palatinose, lactosucrose, glycooligosaccharides, isomaltooligosaccharides, polydextrose, or soybean oligosaccharides.

13. An oligosaccharide composition comprising stachyose, melitriose (raffinose) and verbascose.

14. The oligosaccharide composition of claim 13, further comprising a sufficient amount of an amino acid to reduce or eliminate aldehydic odor of the oligosaccharide composition.

15. The oligosaccharide composition of claim 14, wherein the amino acid is one or more of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or mixtures thereof.

16. The oligosaccharide composition of claim 13, further comprising one or more prebiotics.

17. The oligosaccharide composition of claim 16, wherein the one or more prebiotic comprises fructooligosaccharides, galactooligosaccharides, arabinose, galactose, inulin, raffinose, mannose, lactulose, mannanoligosaccharides, xylooligosaccharides, palatinose, lactosucrose, glycooligosaccharides, isomaltooligosaccharides, polydextrose, or soybean oligosaccharides.

18. The oligosaccharide compositions of claim 1, wherein the stachyose is derived from Chinese herb and/or common vegetable;
preferably, the Chinese herb is selected from Rehmannia Glutinosa and/or Eupatorium Japonicum Thumb; and the common vegetable is selected from Chinese artichoke and/or legume; more preferably, the legume is selected from green beans, soy beans and/or peas.

19. The oligosaccharide compositions of claim 4, wherein the melitriose is derived from Chinese herb and/or edible plant, wherein the Chinese herb is selected from Rehmannia Glutinosa and/or Eupatorium Japonicum Thumb and the edible plant is selected from the group consisting of Chinese artichoke, beans, asparagus, cotton seeds, sugar beet molasses, cabbage, broccoli, Brussel's sprouts, sweet potatoes and/or whole grains.

20. The oligosaccharide compositions of claim 4, wherein the verbascose is derived from Chinese herb and/or edible plant, wherein the Chinese herb is selected from Rehmannia Glutinosa and/or Eupatorium Japonicum Thumb and the edible plant is selected from Chinese artichoke, and/or a legume.
